(19) United States
(12) Patent Application Publication
(10) Pub. No.: US 2022/0160361 A1
(43) Pub. Date: May 26, 2022

(54) EVERSION TOOL AND SIZE GUIDE FOR PERFORMING ARTERIAL MICROVASCULAR ANASTOMOSIS

(71) Applicants: BAXTER INTERNATIONAL INC., Deerfield, IL (US); BAXTER HEALTHCARE SA, Glattpark (Opfikon) (CH)

(72) Inventors: Franklin Sullivan Busch, Cincinnati, OH (US); John Case Coolidge, Ringwood, IL (US); Ben KO, Cincinnati, OH (US); James Douglas Studer, Beldenville, WI (US); Anna Searle, West Lafayette, IN (US)

(21) Appl. No.: 17/425,712

(22) PCT Filed: Jan. 23, 2019
(86) PCT No.: PCT/US2019/014767

§ 371 Date: Jul. 23, 2021

(51) Int. Cl. A61B 17/11; A61B 90/00

(52) U.S. Cl. CPC A61B 17/11; A61B 90/06; A61B 2017/00433; A61B 2017/1121; A61B 2017/1132; A61B 2017/1107

(57) ABSTRACT

An everter device includes a handle and at least one eversion tip. The handle has a first end and a second and each of the at least one eversion tip is coupled to a respective end of the handle. Additionally, the at least one eversion tip has a respective distal end, a respective proximal end, and a respective eversion surface. A sizing device includes a handle and at least one sizing guide. The handle has a first end and a second end. Each of the at least one sizing guide is coupled to a respective end of the handle. Additionally, the at least one sizing guide has a plurality of sizing apertures and associated sizing indicators. The everter device and the sizing device may be provided together as a kit.

### BACKGROUND

Plastic and reconstructive surgery regularly uses free flaps, for example in breast reconstruction. In free flap tissue surgery, a free flap (e.g., tissue and/or muscle and its associated artery and vein) is removed from one part of the body or donor site and is reattached to another part of the body or recipient site. The artery and vein of the transferred tissue and/or muscle are then anastomosed to a native artery and vein in order to achieve blood circulation in the transferred free flap (e.g., tissue and/or muscle).

The anastomosis of the free flap tissue to the native tissue is typically done using microvascular techniques, including under microscopic visualization. In previous years, several surgical instruments and techniques have been developed to aid in anastomosis. One known system for creating an anastomosis is an anastomosis coupler, described in U.S. Pat. No. 7,192,400, the disclosure of which is incorporated herein by reference. This anastomotic coupler is a surgical instrument that allows a surgeon to more easily and effectively join together two blood vessel ends. The coupler involves the use of two fastener portions, in the shape of rings, upon which are secured respective sections of the vessel to be attached. Each fastener portion is also provided with a series of pins, and corresponding holes for receiving those pins, in order to close and connect the portions, and in turn the vessel, together (See FIGS. 8A and 8B).

Microvascular anastomosis is the surgical coaptation of veins and arteries. Microvascular anastomosis of veins is readily accomplished using a microanastomotic coupling device, such as the GEM FLOW COUPLER®, which reduces complication rates, improves patency rates, substantially reduces the time necessary to complete the coaptation compared to manual suturing techniques. However, microanastomosis of arteries is most often accomplished with standard manual suturing techniques because the thick, muscular wall of the arteries precludes use of the current microanastomotic couplers. The thick wall of the artery prevents the tissue of the arterial wall from being stretched over the rings of a coupler. Each microanastomotic coupler ring has a plurality of pins or posts, which are used to secure an everted portion of a vessel segment to the ring. Even after securing one portion of an everted arterial segment to a pin or post (or even a few pins or posts) of a microanastomotic coupler ring, efforts to secure remaining portions of the everted arterial segment to the coupler ring are often complicated by the first portion coming off the previously-secured pin(s) or post(s). Due to the lack of a reliable device or technique to avoid this problem, manual suturing is predominantly used for surgical coaptation of arteries.

However, microscopic manual suturing of arteries can be quite challenging, primarily due to the small size of the vessels and the minimal working space. Since most vessels are only 1 to 3 mm in diameter, the procedure requires the use of a surgical microscope. The sutures are about 70 μm thick and can be difficult to handle and as a result, medical practitioners (e.g., surgeons and surgical residents) must undergo extensive additional training prior to operating on a patient in need of tissue transfer. Moreover, surgeons attempt to limit the recipient site morbidity resulting in small incisions and small areas within which to work. For instance, in microsurgical postmastectomy breast reconstruction, the surgeon may typically work in a surgical field of approximately 3 cm or less. These size constraints may make it difficult for surgeons to maneuver their surgical instruments. Arterial microanastomoses performed by manual suturing take approximately 23.5 minutes in the operating room, versus coaptation times as low as 5 minutes or less that would be possible if a surgeon were using a coupling device.

### SUMMARY

The present disclosure provides improved vessel sizing and vessel eversion systems, devices and methods to improve the coaptation of veins and arteries in arterial microvascular anastomosis procedures.

In a first embodiment, an everter device includes a handle and at least one eversion tip. The handle has a first end and a second and each of the at least one eversion tip is coupled to a respective end of the handle. Additionally, the at least one eversion tip has a respective distal end, a respective proximal end, and a respective eversion surface.

In another example embodiment, the at least one eversion tip is rotatably coupled to the handle.

In another example embodiment, the at least one eversion tip is removably coupled to the handle.

In another example embodiment, the eversion surface includes a piloting region starting at the distal end of the eversion tip, a concave region, a transition region, and a nearly linear region ending at the proximal end of the eversion tip. The concave region connects the piloting region to the transition region, the transition region connects the concave region to the nearly linear region, and the eversion surface transitions from a concave surface to a convex surface in the transition region. Additionally, the eversion tip has a diameter. The diameter of the eversion tip in the concave region increases exponentially as the eversion surface approaches the transition region. The diameter of the eversion tip in the transition region continuously increases through the transition region towards the nearly linear region. The rate of change of the diameter of the eversion tip in the transition region continuously decreases through the transition region towards the nearly linear region, and the diameter of the eversion tip in the nearly linear region continues to increase until reaching the proximal end of the eversion tip.

In another example embodiment, the eversion surface is a curved surface between the distal end and the proximal end of the eversion tip. The curved surface slopes outward toward a shoulder of the eversion tip and then slopes back inward after the shoulder as the curved surface approaches the proximal end.

In another example embodiment, the eversion surface is configured to expand radially outward at the shoulder in response to a compressive force applied on the curved surface. The compressive force is applied toward the proximal end.

In another example embodiment, the eversion tip is made from an elastic material, which is piercable, deformable, or that is adapted to axially recede to accommodate posts or pins of a coupler ring of an anastomosis clamp system.

In another example embodiment, the elastic material is configured to deform or axially recede when the everter device is advanced into contact with posts or pins of a coupler ring.

In another example embodiment, the elastic material is a thermoplastic elastomer.

In another example embodiment, the thermoplastic elastomer is a silicone elastomer.

In another example embodiment, the silicone elastomer is NUSIL 4840 silicone.

In another example embodiment, the eversion tip has a Shore A hardness between 10 and 50.

In another example embodiment, the eversion tip has a Shore A hardness between 30 and 45.

In another example embodiment, the handle includes a plurality of gripping members.

In another example embodiment, the handle includes at least one retention barb configured and arranged to retain the at least one eversion tip on the handle.

In another example embodiment, the retention barb includes a shelf, a trunk and a cap. The trunk is adjacent to the shelf and extends from the shelf to the cap. Additionally, the cap forms a respective end of the handle.

In a second embodiment, a method includes providing a coupler ring on a vessel segment where the coupler ring has a plurality of pins projecting therefrom, such that the securement pins are directed toward a free end of the vessel segment. The method also includes advancing a rotatable eversion tip of an everter device toward the coupler ring and the free end of the vessel segment until a distal end of the eversion tip is received in the free end of the vessel segment and advanced past the coupler ring. Additionally, the method includes continuing to advance the rotatable eversion tip of the everter device toward the coupler ring until the free end of the vessel segment is everted over the coupler ring, applying sufficient force to the everter device to cause the pins of the coupler ring to pierce through the everted free end of the vessel segment, and removing the everter device from the vessel segment.

In a third embodiment, a sizing device includes a handle and at least one sizing guide. The handle has a first end and a second end. Each of the at least one sizing guide is coupled to a respective end of the handle. Additionally, the at least one sizing guide has a plurality of sizing apertures and associated sizing indicators.

In another example embodiment, the at least one sizing guide is removably coupled to the handle.

In another example embodiment, the handle includes a plurality of gripping members.

In another example embodiment, the handle includes at least one retention member configured and arranged to retain the at least one sizing guide on the handle.

In another example embodiment, the retention member is a retention barb.

In another example embodiment, the plurality of sizing apertures have diameters between 0.1 mm and 10.0 mm.

In a fourth embodiment, an eversion kit includes an everter device and a sizing device. The everter device includes a first handle having a first end and a second end, and at least one eversion tip. Each of the at least one eversion tip is coupled to a respective end of the handle. The at least one eversion tip has a respective distal end, a respective proximal end and a respective eversion surface. The sizing device includes a second handle having a first end portion and a second end portion, and at least one sizing guide. The at least one sizing guide is coupled to a respective end portion of the handle. Additionally, the at least one sizing guide has a plurality of sizing apertures and associated sizing indicators.

In another example embodiment, the at least one eversion tip of the everter device is rotatably coupled to the handle.

In another example embodiment, the at least one eversion tip of the everter device is removably coupled to the handle.

In another example embodiment, the eversion surface of the at least one eversion tip of the everter device is a curved surface between the distal end and the proximal end of the eversion tip. The curved surface slopes outward toward a shoulder of the eversion tip and then slopes back inward after the shoulder as the curved surfaces approaches the proximal end of the eversion tip.

In another example embodiment, the eversion tip is made from an elastic material, which is piercable, deformable, or that is adapted to axially recede to accommodate posts or pins of a coupler ring of an anastomosis clamp system.

In another example embodiment, the eversion tip has a Shore A hardness between 10 and 50 and preferably a Shore A hardness between 30 and 45.

In another example embodiment, the first handle and the second handle both include a plurality of gripping members.

In another example embodiment, the first handle includes at least one retention barb configured and arranged to retain the at least one eversion tip on the first handle.

In a fifth embodiment, a method includes determining a size of a vessel segment and providing a coupler ring on a vessel segment. The coupler ring has a plurality of pins projecting therefrom, such that the securement pins are directed toward a free end of the vessel segment. The method also includes advancing an eversion tip of an everter device toward the coupler ring and the free end of the vessel segment until a distal end of the eversion tip is received in the free end of the vessel segment and advanced past the coupler ring, continuing to advance the eversion tip of the everter device toward the coupler ring until the free end of the vessel segment is everted over the coupler ring, applying sufficient force to the everter device to cause the pins of the coupler ring to pierce through the everted free end of the vessel segment, and removing the everter device from the vessel segment.

It is accordingly an advantage of the present disclosure to improve coaptation of veins and arteries in arterial microvascular anastomosis procedures

It is another advantage of the present disclosure to improve vessel wall capture and retention on pins of a ring coupler device used for a microvascular anastomosis procedure.

It is another advantage of the present disclosure to improve vessel wall eversion when performing a microvascular anastomosis procedure when using a ring coupler device.

It is a further advantage of the present disclosure to provide reliable vessel sizing device and methods.

It is yet a further advantage of the present disclosure to reduce vessel wall tearing and failure when performing a microvascular anastomosis procedure when using a ring coupler device.

It is still another advantage of the present disclosure to reduce coaptation times when performing arterial microanastomosis procedures.

Additional features and advantages of the disclosed vessel sizing and vessel eversion systems, devices and methods are described in, and will be apparent from, the following Detailed Description and the Figures. The features and advantages described herein are not all-inclusive and, in particular, many additional features and advantages will be apparent to one of ordinary skill in the art in view of the figures and description. Also, any particular embodiment does not have to have all of the advantages listed herein. Moreover, it should be noted that the language used in the specification has been principally selected for readability and instructional purposes, and not to limit the scope of the inventive subject matter.

### BRIEF DESCRIPTION OF THE FIGURES

FIG. 1A is a perspective view of an everter device according to an example embodiment of the present disclosure.

FIG. 1B is a top view of an everter device according to an example embodiment of the present disclosure.

FIG. 1C is a side view of an everter device according to an example embodiment of the present disclosure.

FIG. 2A is a perspective view of a handle of an everter device according to an example embodiment of the present disclosure.

FIG. 2B is a top view of a handle of an everter device according to an example embodiment of the present disclosure.

FIG. 2C is a side view of a handle of an everter device according to an example embodiment of the present disclosure.

FIG. 2D is a cross-sectional view of a handle of an everter device along line 2D-2D of FIG. 2B according to an example embodiment of the present disclosure.

FIG. 3A is a partial plan view of a retention barb of a handle of an everter device according to an example embodiment of the present disclosure.

FIG. 3B is a partial plan view of a retention barb of a handle of an everter device according to an example embodiment of the present disclosure.

FIG. 4A is a partial plan view of an alternative retention barb of a handle of an everter device according to an example embodiment of the present disclosure.

FIG. 4B is a partial plan view of an alternative retention barb of a handle of an everter device according to an example embodiment of the present disclosure.

FIG. 5A is a perspective view of an eversion tip of an everter device according to an example embodiment of the present disclosure.

FIG. 5B is a perspective view of an eversion tip of an everter device according to an example embodiment of the present disclosure.

FIG. 5C is a side view of an eversion tip of an everter device according to an example embodiment of the present disclosure.

FIG. 5D is a cross-sectional view of an eversion tip along line 5D-5D of FIG. 5C according to an example embodiment of the present disclosure.

FIG. 5E is a cross-sectional view of an eversion tip along line 5D-5D of FIG. 5C according to an example embodiment of the present disclosure.

FIG. 5F is a side view of an eversion tip with example dimensions according to an example embodiment of the present disclosure.

FIG. 5G is a side view of an eversion tip with example dimensions according to an example embodiment of the present disclosure.

FIG. 6A is a perspective view of a sizing device according to an example embodiment of the present disclosure.

FIG. 6B is a top view of a sizing device according to an example embodiment of the present disclosure.

FIG. 6C is a side view of a sizing device according to an example embodiment of the present disclosure.

FIG. 6D is a cross-sectional view of a sizing device along line 6D-6D of FIG. 6C according to an example embodiment of the present disclosure.

FIG. 6E is a cross-sectional view of a sizing device along line 6E-6E of FIG. 6C according to an example embodiment of the present disclosure.

FIG. 7A is a partial plan view of a sizing guide of a sizing device according to an example embodiment of the present disclosure.

FIG. 7B is a partial plan view of a sizing guide of a sizing device according to an example embodiment of the present disclosure.

FIG. 8A is a side view of a coupler ring positioned over a vessel.

FIG. 8B is a cross-sectional view of FIG. 8A along the centerline of the vessel.

FIG. 9A is a perspective view of an everter device and a coupler ring positioned over a vessel according to an example embodiment of the present disclosure.

FIG. 9B is a side view of an everter device everting a vessel onto a coupler ring positioned over a vessel according to an example embodiment of the present disclosure.

FIG. 10 is a table of retention barb designs.

FIG. 11 is a table of retention barb designs.

FIG. 12A is a table of eversion tip designs.

FIG. 12B includes charts of average hardness and average number of pins captured from the eversion tip designs in FIG. 12A.

FIG. 13A is a table of eversion tip designs.

FIG. 13B is a chart of silicone tip separation forces for eversion tip designs of FIG. 13A.

FIG. 14A is a table of eversion tip and retention barb assembly designs.

FIG. 14B is a chart of silicone tip retention strength for eversion tip and retention barb designs of FIG. 14A.

FIG. 15A is a table of eversion tip designs.

FIG. 15B is a table of eversion tip designs.

FIG. 15C is a table of eversion tip designs.

FIG. 15D is a table of eversion tip designs.

FIG. 15E is a graph of change in displacement v. change in force for eversion tip designs of FIGS. 15A to 15D.

FIG. 16A is an alternative embodiment of a combination everter and sizing device.

FIG. 16B is an alternative embodiment of a combination everter and sizing device.

### DETAILED DESCRIPTION OF EXAMPLE EMBODIMENTS

As discussed above, vascular monitoring system, device and method are provided to reduce vessel wall tearing and failure and improve vessel wall eversion and vessel wall capture and retention on pins of a ring coupler device when performing a microvascular anastomosis procedure. Additionally, the systems, devices and methods disclosed herein are provided to enable reliable vessel sizing as to reduce coaptation times when performing arterial microanastomosis procedures. Arterial microanastomoses performed by manual suturing take approximately 23.5 minutes in the operating room, versus coaptation times as low as 5 minutes or less when successfully using a coupling device. Successful using of a coupling device often requires accurately determining vessel sizes and properly and efficiently everting the vessel wall to ensure vessel wall capture and retention on pins of the ring coupler.

In an effort to promote efficiency in the execution of an arterial microanastomosis procedure, it is found that the challenges presented by the relatively thick wall of an artery (as compared to the wall thickness of a vein) can be mitigated by applying uniform support to a region of an arterial segment just behind, and/or within, a coupler ring of an anastomosis clamp system such as the such as the GEM FLOW COUPLER® of Synovis Micro Companies. Example embodiments of everter devices and sizing devices are described in further detail below.

### Everter Device

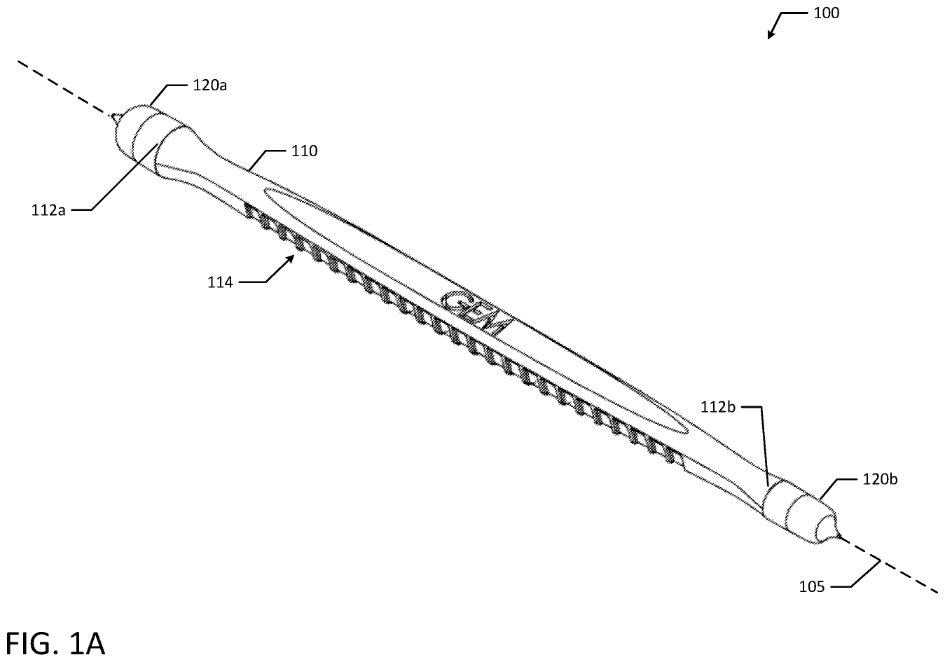

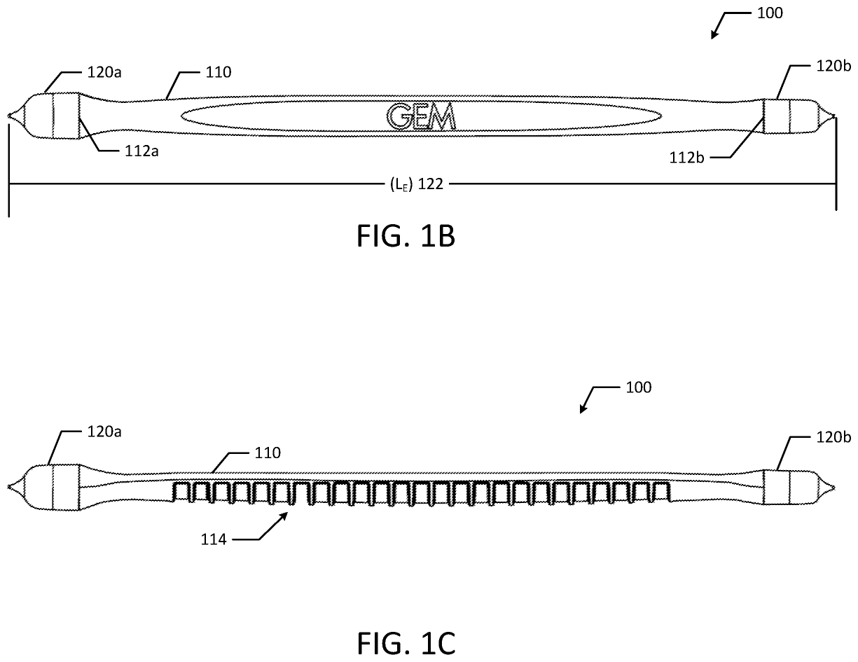

As illustrated in FIGS. 1A, 1B and 1C, an everter device 100 includes one or more eversion tips (e.g., large eversion tip 120a and small eversion tip 120b, hereinafter referred to generally as inversion tip 120) positioned on a handle 110. In an example, eversion tip 120a is positioned on a first end of handle 110 and eversion tip 120a is positioned on a second end of handle 110. In an example, eversion tip 120 may be overmolded on handle 110.

Handle 110 may include a first shoulder 112a near the first end a second shoulder 112b near the second end (hereinafter referred to generally as shoulder 112). Eversion tip(s) 120 may be substantially flush with the edge of shoulder(s) 112 such that the eversion tip(s) are in contact with shoulder(s) 112 or are in close proximity with shoulder(s) 112 with a small gap (e.g., a gap of approximately 0.006 inches or 0.15 mm). Even if eversion tip(s) 120 are in contact with shoulder(s) 112, the eversion tip(s) 120 may still be capable of rotating about central axis 105.

In an example, the length (L_{E}) 122 of the everter device 100 may be between 4.0 inches to 7.0 and may preferably be approximately 5.7 inches (144.78 mm). The size, shape and dimensions (e.g., length) of everter device 100 may be adapted to provide sufficient surface area for gripping and handling by a medical practitioner as well as being optimized for use in small surgical fields (e.g., surgical fields of approximately 3 cm or less).

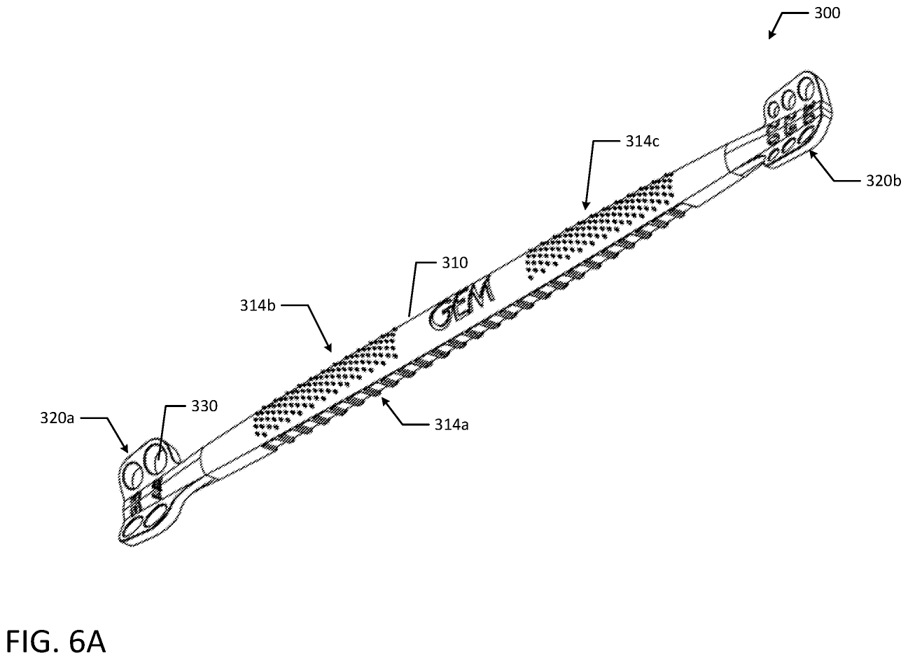

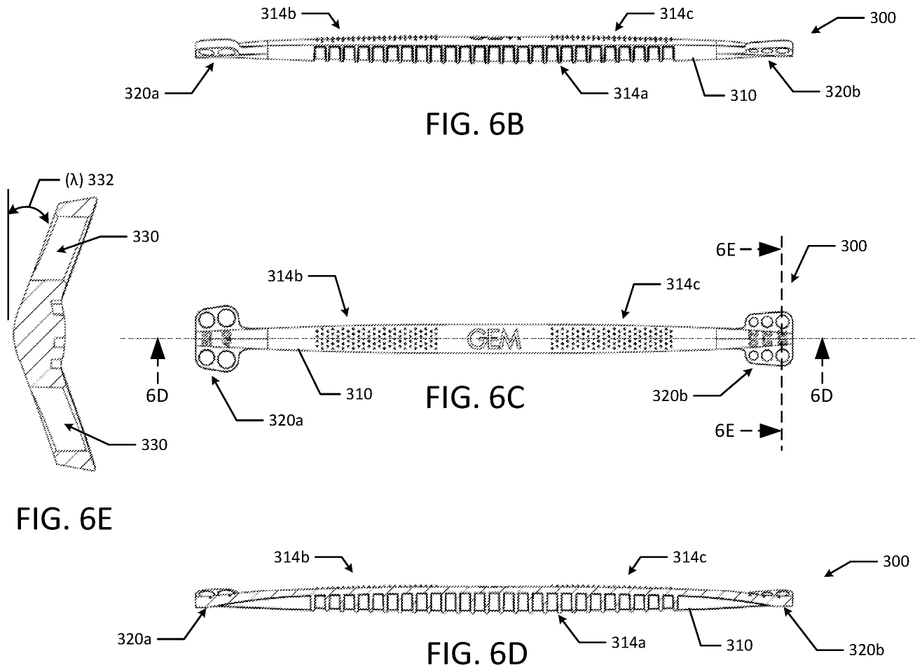

In an example, handle 110 may include gripping members 114. For example, the bottom of handle 110 may include gripping member 114, such as a plurality of ribs or ridges to provide the user, such as a medical practitioner, additional gripping and handling features on handle 110. Additionally, gripping members 114 may comprise a plurality of projections or bumps to provide better gripping and handling characteristics of everter device 100 (similar to gripping members 314b and 314c of FIG. 6A). It should be appreciated that other gripping means may be included on handle 110 (e.g., knurling, grips, ridges or the like) to provide additional gripping and handling characteristics to everter device 100. The gripping members 114 may advantageously aid the user, such as a medical practitioner, when the user is handling and/or manipulating the everter device 100 to evert a vessel during microvascular anastomoses.

Handle 110 may be formed of a rigid or semi-rigid material, such as thermoplastic, that is adapted to provide support for eversion tips 120 while preventing excessive deformation during use. For example, handle 110 may be made from MAKROLON 2458 polycarbonate. In another example, the exterior of the everter device 100 may be made substantially of or entirely made of pierceable material. For example, both the handle 110 and eversion tips 120 may be formed of a pierceable material and the everter device 100 may further include a supporting rod or other internal structural support member. The supporting rod may be made of a rigid or semi-rigid material that is adapted to support eversion tops 120 while preventing excessive deformation during use.

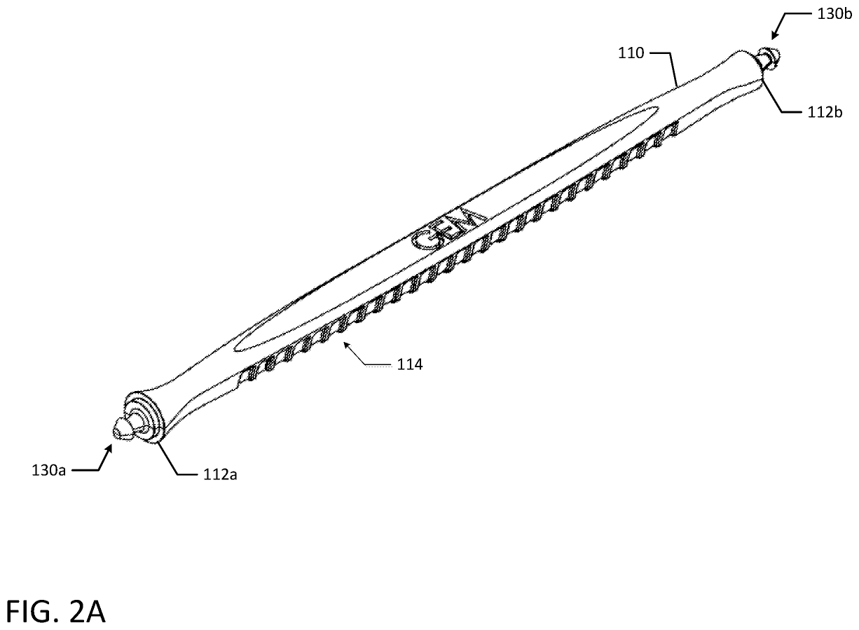

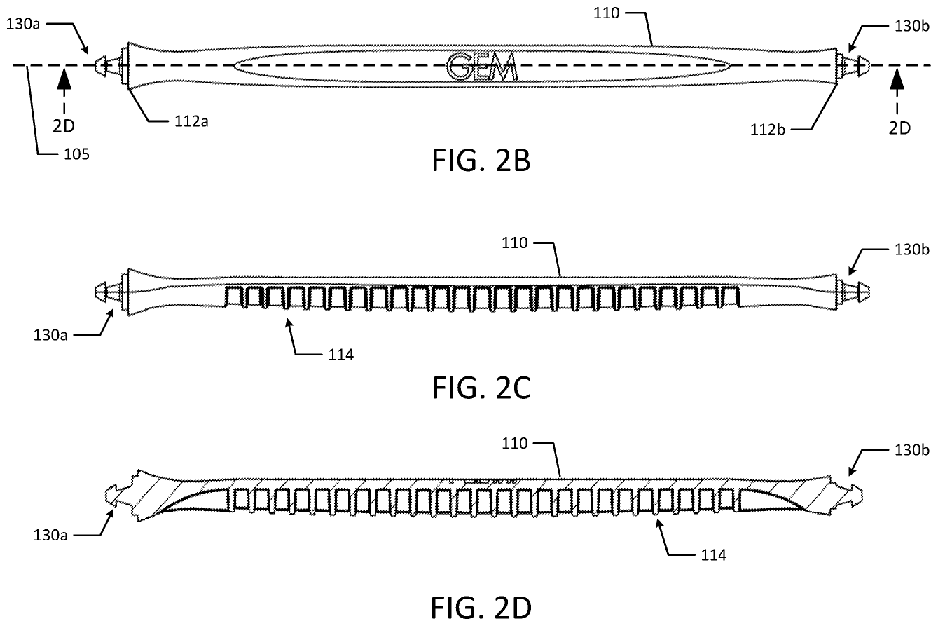

As illustrated in FIGS. 2A, 2B, 2C and 2D, handle 110 may include retention barbs (e.g., barb 130a at the first end of handle 110 and barb 130b at the second end of handle 110, hereinafter referred to generally as barb(s) 130). Each barb 130 may be sized and shaped such that it retains eversion tip 120 on handle 110. For example, eversion tip(s) 120 may be press-fit onto barb(s) 130 of handle 110 such that they are removably retained on handle 110. As discussed above, in an example, the eversion tip(s) 120 may be rotatably coupled to handle 110 via barb(s) 130. For example, the retention barbs 130 may include retention features (e.g., cap 170 discussed in more detail below) to prevent eversion tips 120 from being easily removed from (e.g., pulled off of) handle 110 while still providing enough internal clearance such that eversion tips 120 can rotate freely about central axis 105 of retention barbs 130. Specifically, retention barbs 130 may maintain the axial position (e.g., prevent longitudinal displacement along axis 105) of eversion tips 120 unless a sufficient pulling force is applied by a user to remove the eversion tips 120 from handle 110.

Retention barbs 130 may be formed as a single piece with handle 110. In another example, retention barbs 130 may be threadingly connected to handle 110 such that different barb geometries may be interchangeably attached to handle 110.

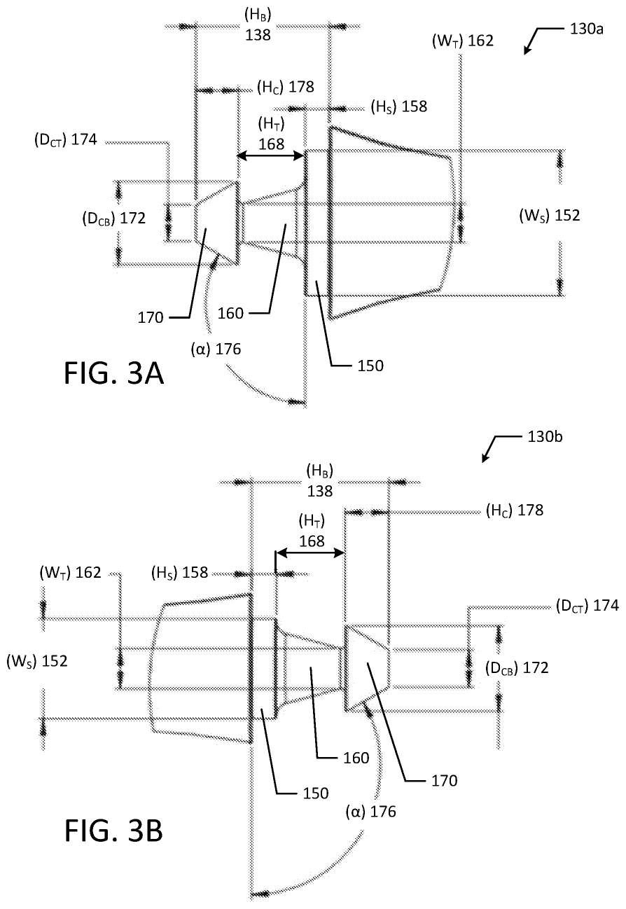

FIG. 3A illustrates a partial plan view of barb 130a of everter handle 110 and FIG. 3B illustrates a partial plan view of barb 130b of everter handle 110. As discussed above, each barb 130 may be sized and shaped such that it retains eversion tip 120 on handle 110. In an example, barb 130 may have a barb height (H_{B}) 138. As illustrated in FIGS. 3A and 3B, barb(s) 130 may include a shelf 150, a trunk 160 and a cap 170. Shelf 150 may be positioned adjacent shoulder 112 and may have a shelf width (W_{S}) 152 and a shelf height (H_{S}) 158. In an example, shelf 150 may be a cylindrical shelf where the shelf width (W_{S}) is equivalent to the shelf diameter. Trunk 160 may be positioned between shelf 150 and cap 170 and the trunk 160 may gradually decrease in diameter between shelf 150 and cap 170 at an angle (α). Additionally, the end of the trunk 160 adjacent cap 170 may have a trunk width (W_{T}) 162 and a trunk height (H_{T}) 168. In an example, trunk 150 may be a cylindrical, conical, or frustoconical trunk where the trunk width (W_{T}) is equivalent to the trunk diameter. Cap 170 may be positioned adjacent trunk 160 opposite shelf 150. The cap 170 may similarly have a cylindrical, conical, or frustoconical shape with a cap base and a cap top. As the cap 170 is wider than trunk 160, cap 170 acts as a retaining feature that resists and prevents longitudinal displacement of eversion tip 120 and maintains the axial position of eversion tip 120 along axis 105 to prevent eversion tips 120 from pulling away from handle 110 during use. The cap 170 may gradually decrease in diameter between the cap base, which interfaces with trunk 160 and the cap top. For example, the cap base may have a diameter (D_{CB}) 172 and the cap top may have a diameter (D_{CT}) 174. In an example, the dimeter may change in a linear profile at an angle (α). Additionally, cap 170 may have a cap height (H_{C}) 178.

The cylindrical, conical, and/or frustoconical profiles of the various sections (e.g., shelf 150, trunk 160 and cap 170) of retention barb 130 advantageously retains eversion tips 120 on handle 110 while still allowing eversion tips 120 to rotate. As discussed in more detail below, rotation of eversion tips 120 advantageously reduces the occurrence and/or prevents eversion tip 120 from bending and deforming pins or posts of a ring coupler.

As illustrated in FIG. 3A and 3B, the barb height 138 is equivalent to the sum of the shelf height 158, the trunk height 168 and the cap height 178 (e.g., H_{B}=H_{D}+H_{T}+H_{C}). In an example embodiment, the barb height (H_{B}) 138 may be approximately 0.217 inches (5.50 mm), the shelf height (H_{S}) 158 may be approximately 0.039 inches (1.00 mm), the trunk height (H_{T}) 168 may be approximately 0.108 inches (2.75 mm) and the cap height (H_{C}) 178 may be 0.069 inches (1.75 mm). Additionally, the shelf width (W_{S}) 152 may be approximately 0.236 inches (6.00 mm), the trunk width (W_{T}) 162 may be approximately 0.063 inches (1.61 mm) and the cap may have a base diameter (D_{CB}) 172 and a top diameter (D_{CT}) 174 of approximately 0.15 inches (3.43 mm) and 0.059 inches (1.50 mm) respectively. Additionally, the shelf width (W_{S}) 152 may be approximately 0.236 inches (6.00 mm). In an example, angle (α) 176 may be approximately 120 degrees.

The dimensions above may be adjusted to increase or decrease stiffness imparted to eversion tip 120. Additionally, the dimensions above may be adjusted to improve the engagement between barb 130 and eversion tip 120 to increase the required pulling force to remove eversion tip 120 or to provide additional friction so there is more resistance to eversion tip 120 rotation. Additionally, dimensions may be adjusted to lessen the engagement between barb 130 and eversion tip 120 such that the pulling force is decreased or to reduce friction and lessen the resistance to eversion tip 120 such that eversion tip 120 rotates more freely. In other examples, shelf width (W_{S}) 152 may be approximately 0.220 inches (5.58 mm) or approximately 0.141 inches (3.58 mm).

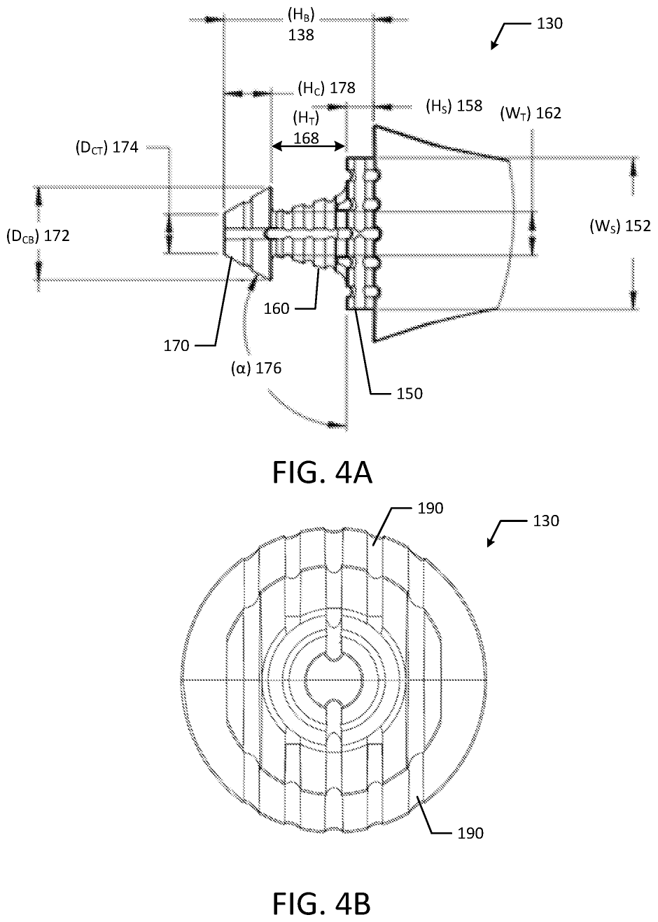

FIG. 4A and FIG. 4B illustrate partial plan views of an alternative embodiment of barb(s) 130 of everter handle 110. As discussed above, each barb 130 may be sized and shaped such that it retains eversion tip 120 on handle 110. Alternative embodiment of barb(s) 130 illustrated in FIGS. 4A and 4B may include the same features (e.g., shelf 150, trunk 160 and cap 170) of the barb(s) 130 illustrated in FIGS. 3A and 3B. However, the alternative barb(s) 130 may include channels or grooves 190 in the shelf 150, trunk 160 and cap 170. For example, channels or grooves 190 may be approximately 0.016 inches wide and approximately 0.008 inches deep. In an example, channels or grooves 190 may reduce the contact surface area between the barb(s) 130 and respective eversion tip(s) 120 thereby reducing friction between the barb(s) and eversion tip(s) 120 and providing enhanced rotation such that eversion tips 120 can rotate freely about central axis 105 of retention barbs 130. In an alternative embodiment, the barb(s) 130 may be oversized such that the channels or grooves 190 provide additional engagement surfaces between barb 130 and eversion tip 120 to enhance eversion tip 120 retention.

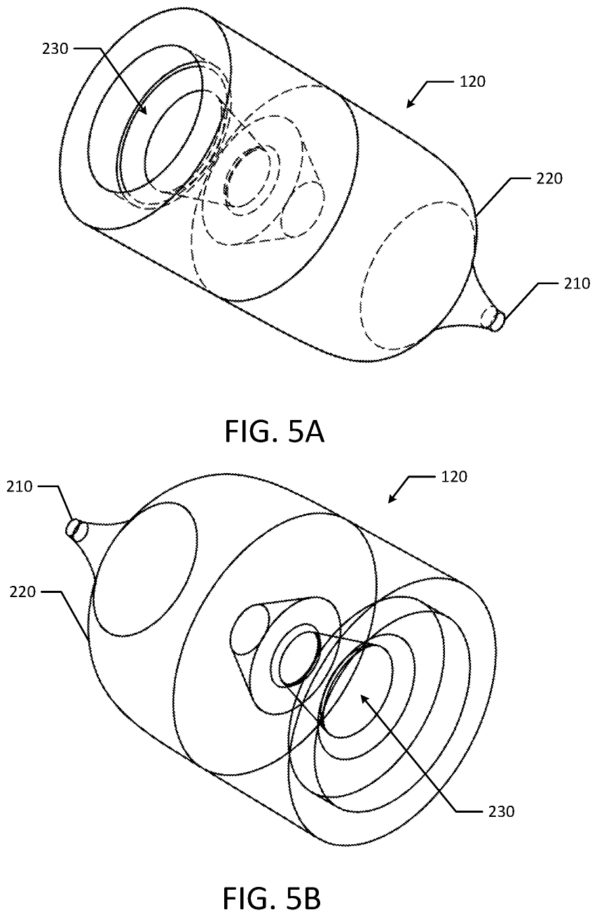

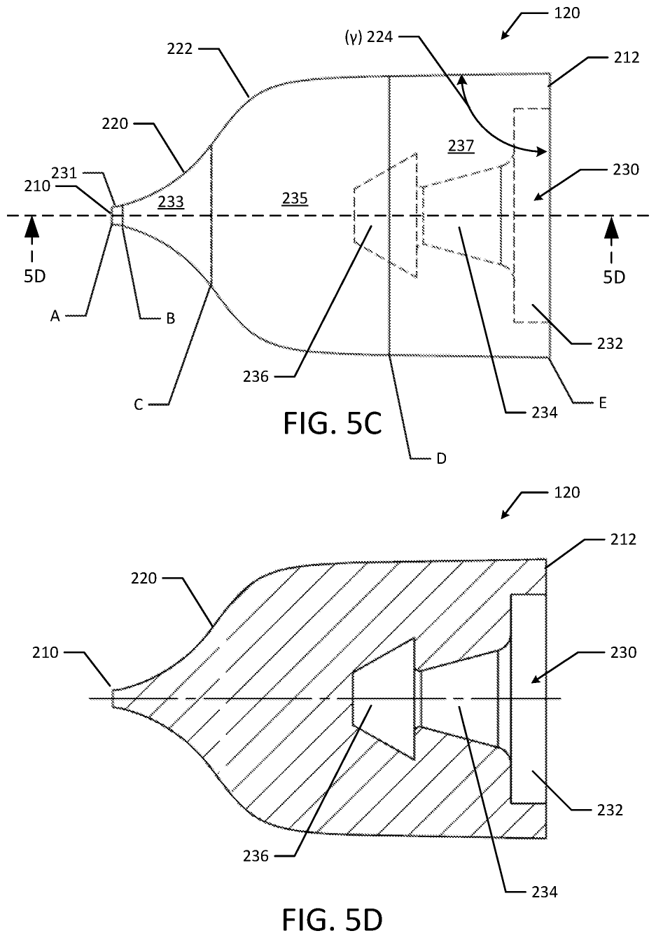

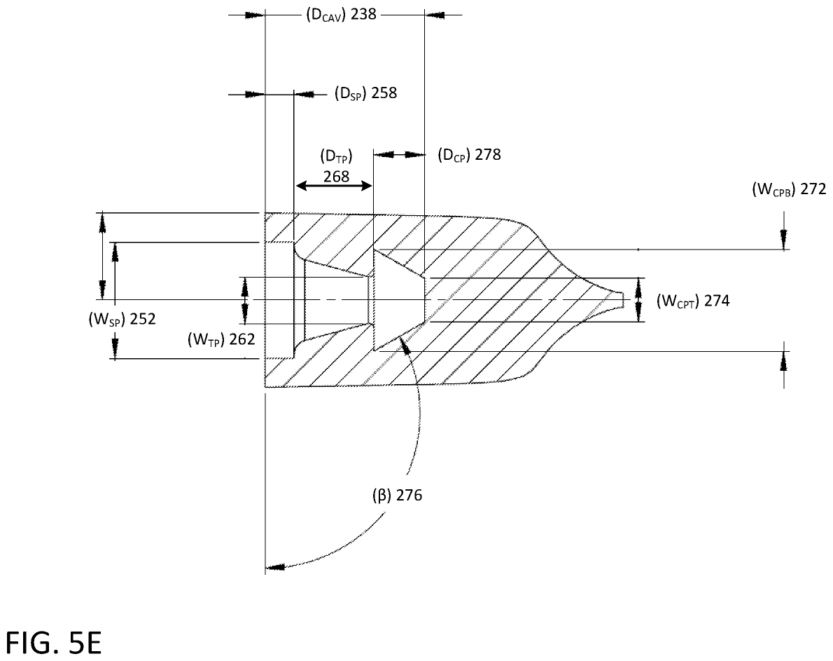

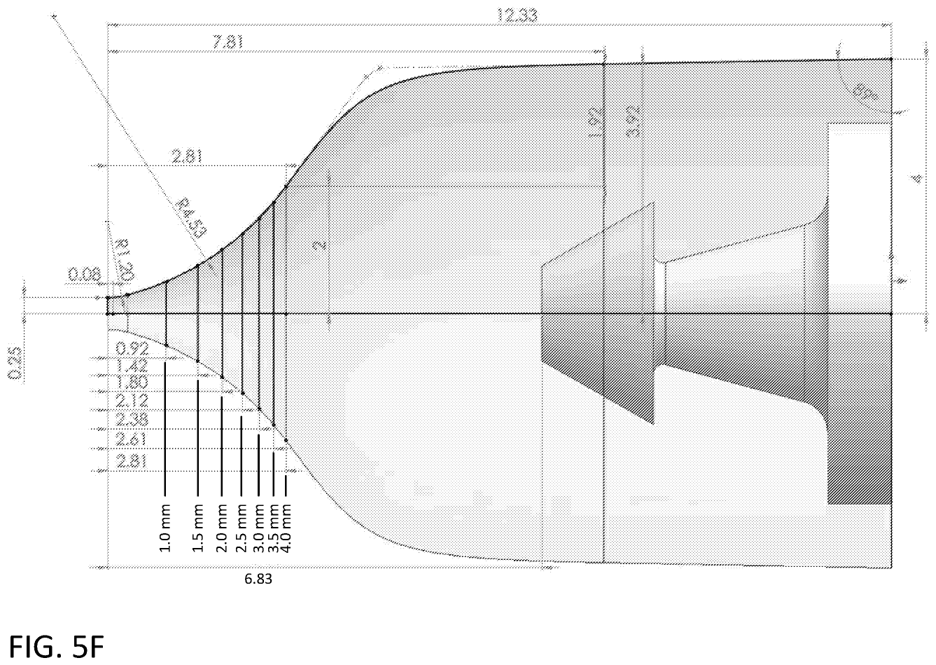

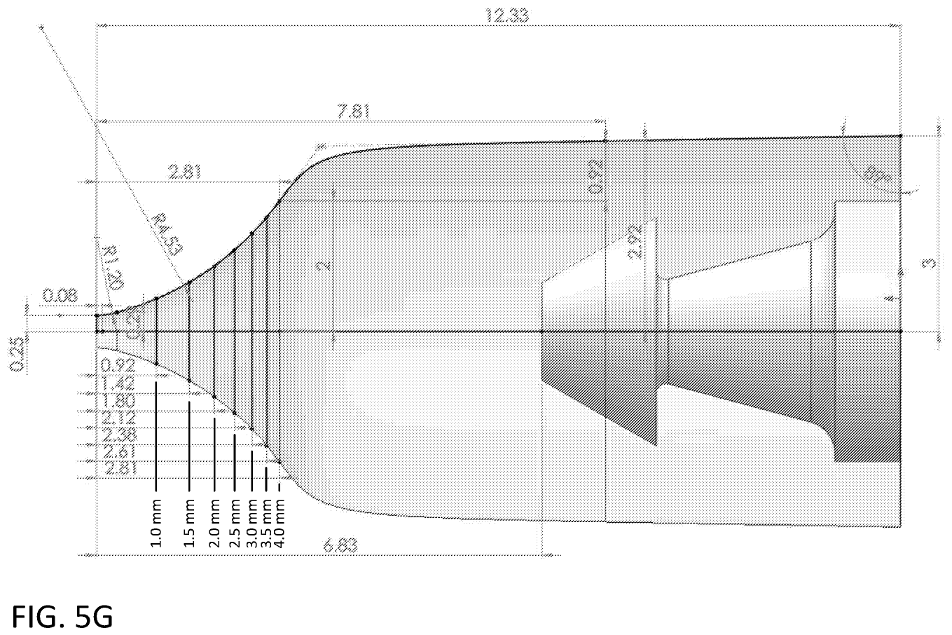

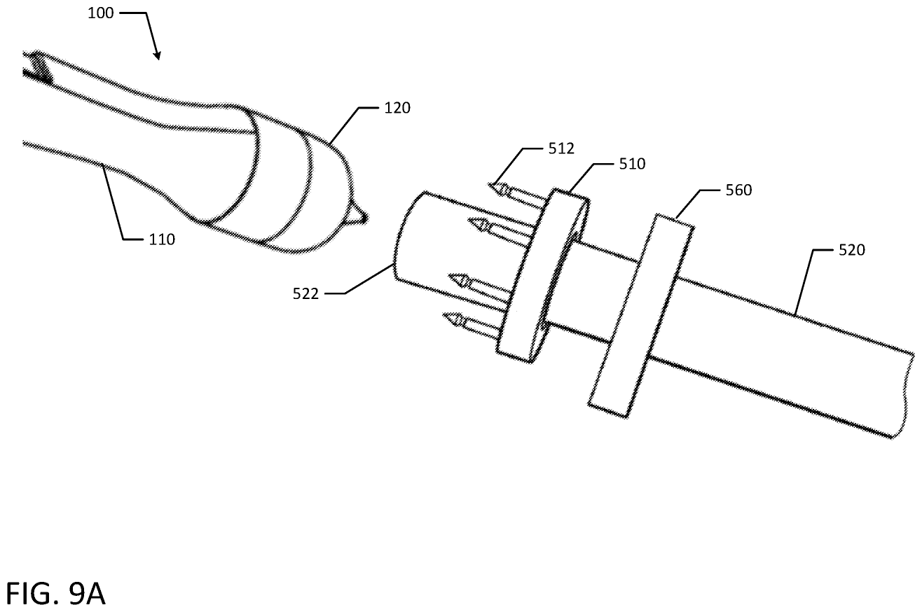

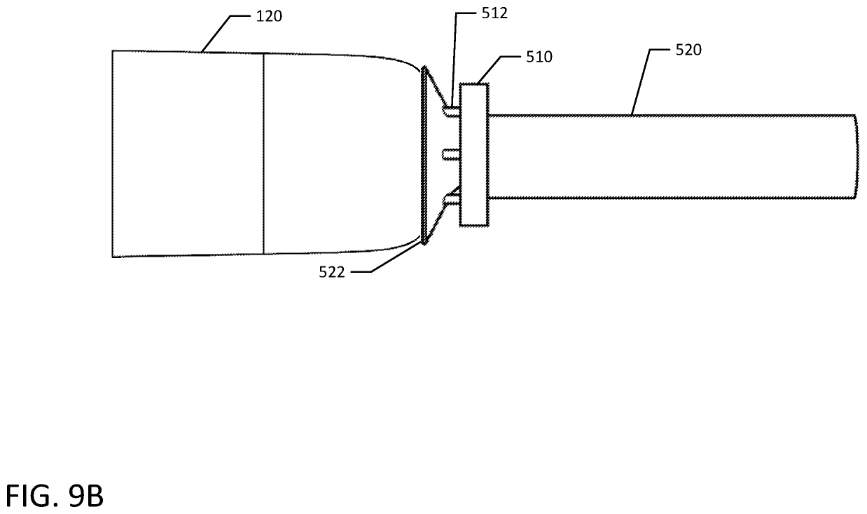

FIGS. 5A, 5B, 5C and 5D illustrate an example embodiment of eversion tip 120. In an example, eversion tip 120 may have a Shore A hardness between 10 and 50 and more preferably a Shore A hardness between 30 and 45. Each eversion tip 120 may be formed of a flexible and/or pierceable material such as a thermoplastic elastomer or silicone rubber. By forming each eversion tip 120 of a flexible and/or pierceable material, the everter device 100 is adapted to allow coupler pins (see FIGS. 9A and 9B) to pierce through the outer eversion surface of ever tip 120 without causing significant deformation of the coupler pins. Additionally, by forming each eversion tip 120 from a flexible material, the eversion tip may advantageously deform, thereby minimizing damage to the intima of the vessel. Eversion tips 120 of everter device 100 may be deformable by a user, such as a medical practitioner, which aids in manipulating the everter device 100 to conform to the corresponding shape of small and/or hard to reach anatomical locations. Arteries typically involved in microvascular anastomosis generally have a diameter ranging from 1 mm to 4 mm and the eversion tip 120 may be sized and shape to accommodate arteries within that size range.

Furthermore, eversion tips 120 may deform to assist with vessel eversion as the deformation causes the eversion tip 120 to compress slightly and “balloon” or “mushroom” out thereby pressing against the internal vessel wall and causing the vessel to further evert. The “ballooning” and/or “mushrooming” of eversion tip 120 is a function of eversion tip geometry, eversion tip material selection, retention barb geometry and retention barb material selection. For example, retention barb geometry and more specifically the distance for the tip of retention barb 130 to the distal end 210 of eversion tip 120 may affect the overall stiffness and/or flexibility of eversion tip 120 thereby affecting how much or how little “ballooning” and/or “mushrooming” takes place as the eversion tip 120 is pushed into a vessel and against a ring coupler.

In an example, eversion tip 120 may be made from NUSIL 4840 silicone. In another example, the eversion tip 120 may be transparent or translucent to provide additional visibility to the user, such as a medical practitioner, when using the everter device 100 to evert a vessel wall onto a coupler ring of an anastomosis clamp system.

Each eversion tip 120 may include a distal end 210 and an eversion surface 220. Additionally, at the proximal end or base 212, eversion tip 120 includes a barb cavity 230. Eversion tip 120 may come to a tip or point at distal end 210, which may have a blunt end (as illustrated in FIG. 5C) or may have a rounded end. From the distal end 210, the eversion surface 220 may slope outward toward shoulder 222 and then slope back inward as it approaches proximal end or base 212, similar to a hyperbolic tangent function. For example, the profile of the eversion surface 220 may be similar to an x-y plot of a hyperbolic tangent function where (−∞) on the x-axis of the plot corresponds to the shape of distal end 210. The profile or contour of the eversion surface 220 follow the shape of the hyperbolic tangent function and as the plot approaches (+∞) on the x-axis the shape or contour of the plot corresponds to where the eversion surface 220 meets the proximal end or base 212). In an example, only a portion of the outside surface of eversion tip 120 may be an eversion surface 220. A portion of the outside surface of eversion tip 120 may have a linear profile such that a portion of the outside surface slopes inward at a constant angle (γ) 224.

The eversion surface 220 may include a piloting region 231 between reference planes “A” and “B”, a concave region 233 between reference planes “B” and “C”, a transition region 235 between reference planes “C” and “D”, and a nearly linear region 237 between reference planes “D” and “E”. The piloting region 231 may have a nearly linear slope as the radius of the eversion surface 220 increases from reference plane “A” to reference plane “B”. As the eversion surface 220 continues beyond the piloting region 231, the radius continues to increase exponentially creating a concave surface in the concave region 233 to reference plane “C”. As illustrated in FIG. 5C, the radius of the eversion surface 220 has the highest rate of change or increase at reference plane “C”. In the transition region 235, the radius of the eversion surface 220 continues to increase, but at a lesser rate and the eversion surface 220 transitions from a convex surface to a concave surface near shoulder 222. After shoulder 222, the rate of change continues to decrease until the slope becomes nearly linear at reference plane “D”. Between reference plane “D” and reference plane “C”, the eversion surface enters the nearly linear region 237 where the radius continues to increase at a nearly constant slope defined by angle (γ) 224.

As illustrated in FIG. 5F, the diameter of the eversion surface 220 starts at 0.5 mm at the distal end 210 or start of the piloting region 231 at reference plane “A” and then the diameter increases at an exponential rate in the direction of reference plane “B”. The distal end 210 or start of the piloting region 231 may include a non-pointed or blunt end so that the eversion surface 220 does not cause a traumatic entry to a vessel. The total width or starting diameter of 0.5mm advantageously enables the eversion tip 120 to pilot the smallest vessels indicated for use with the smallest corresponding coupler rings 510. For example, the diameter increases at an exponential rate from 1.0 mm to 4.0 mm in the concave region 233. The distance between each 0.5 mm increase in diameter lessens and lessens as the eversion surface 220 extends from the piloting region 231 through the concave region 233 to the start of the transition region 235 at reference plane “C”. For example, the distance between the 1.0 mm diameter and the 1.5 mm diameter is 0.5 mm (e.g., 1.42 mm minus 0.92 mm), the distance between the 1.5 mm diameter and the 2.0 mm diameter is 0.38 mm (e.g., 1.80 mm minus 1.42 mm), the distance between the 2.0 mm diameter and the 2.5 mm diameter is 0.32 mm, the distance between the 2.5 mm diameter and the 3.0 mm diameter is 0.26 mm, the distance between the 3.0 mm diameter and the 3.5 mm diameter is 0.23 mm, and the distance between the 3.5 mm diameter and the 4.0 mm diameter is 0.20 mm. Specifically, in the illustrated example, the distance between each 0.5 mm diameter increases lessens from 0.5 mm to 0.20 mm as the eversion surface 220 extends from the piloting region 231 through the concave region. In an example, the concave region 233 may have a radius of curvature of 4.53 mm. Additionally, the piloting region 231 may have a radius of curvature of 1.20 mm. The radius of curvature provides a consistent rate of change for the eversion surface 220 from a diameter of small vessels to larger vessels in a short transition length, but the transition is also gradual enough to prevent pushing or over-stretching the vessel.

The diameter of the eversion surface 220 then increases at a slower and slower rate and transitions from a concave to a convex surface at shoulder 222. In the illustrated example, the transition region 235 may be approximately 5 mm deep. For example, the transition region may start (e.g., reference plane “C”) at approximately 2.81 mm from the distal end 210 and may end (e.g., reference plane “D”) at approximately 7.81 mm from the distal end 210 (e.g., reference plane “A”). As the eversion surface approaches the proximal end 212 of the eversion tip 120 at reference plane “E”, the diameter is increasing in a linear fashion. In an example, the diameter at the proximal end 212 is 8.0 mm. Additionally, in the illustrated example, the angle (γ) 224 of the nearly linear region 237 may be 89 degrees.

FIG. 5G illustrates another example embodiment of an eversion tip 120 with a smaller diameter at the proximal end 212. Similar to the illustration in FIG. 5F, the diameter of the eversion surface 220 starts at 0.5 mm at the distal end 210 or start of the piloting region 231 at reference plane “A” and then the diameter increases at an exponential rate in the direction of reference plane “B”. For example, the diameter increases at an exponential rate from 1.0 mm to 4.0 mm in the concave region 233. The distance between each 0.5 mm increase in diameter lessens and lessens as the eversion surface 220 extends from the piloting region 231 through the concave region 233 to the start of the transition region 235 at reference plane “C”. Then, the diameter of the eversion surface 220 then increases at a slower and slower rate and transitions from a concave to a convex surface at shoulder 222. As the eversion surface approaches the proximal end 212 of the eversion tip 120 at reference plane “E”, the diameter is increasing in a linear fashion. In the illustrated example, the diameter at the proximal end 212 is 6.0 mm. Additionally, in the illustrated example, the angle (γ) 224 of the nearly linear region 237 may be 89 degrees. In the illustrated example, the curve at the shoulder 222 may be tangent to straight lines extended from the nearly linear region 237 and the end of the 4.53 mm radius of curvature from the concave region 233.

The exponential like curve of the concave region 233 and the abrupt rate of change of the radius after the shoulder 222 advantageously provides a shelf-like surface to accept pins or posts 512 of a coupler ring 510 that pierce through eversion tip 120. For example, as illustrated in FIG. 5F, at reference plane “D”, the eversion surface 220 may have a diameter of 7.84 mm, which advantageously provides a sufficient “shoulder” or shelf-like surface that overlaps pins or posts 512 of a coupler ring 510. In an example with a 4 mm coupler ring 510, a pin or post may enter the eversion surface near reference plane “C” where the eversion surface has a diameter of approximately 4 mm to ensure that the eversion tip captures each of the pins or posts 512 (e.g., eight pins in total) with a single axial motion by the user.

In the examples illustrated in FIGS. 5G and 5F, the distance from the cap portion 236 (illustrated in FIG. 5C) to the distal end 210 is 6.83 mm. The distance from the cap portion 236 to the distal end 210 is adapted to allow the user to control the eversion surface 220 when piloting and everting a vessel, but also to allow for a mushroom effect (e.g., the tip “balloons” or “mushrooms” out) such that the diameter of the eversion surface 220 grows in compression and aids in everting the artery or vessel over pins or post 512 while being atraumatic. For example, longer eversion tips 210 may provide additional mushroom effect but may offer less control to the user. Similarly, shorter eversion tips 210 may provide more control to a user, but may not sufficiently mushroom out in compression.

As illustrated in FIGS. 5C and 5D, barb cavity 230 may include a shelf portion 232, a trunk portion 234 and a cap portion 236 to accommodate corresponding portions of barb 130 (e.g., shelf 150, trunk 160 and cap 170). Shelf portion 232 may be positioned adjacent trunk portion 234, which may be positioned adjacent cap portion 236. The barb cavity 230 may be sized and shape to retrain eversion tip 120 on retention barb 130. In an example, barb cavity 230 may be adapted to allow eversion tip 120 to rotate while being retained on retention barb 130 by providing enough clearance to rotate, but still remain physically attached to everter handle 110.

In an example, barb cavity 230 may have a cavity depth (D_{CAV}) 238. As illustrated in FIG. 5E, shelf portion 232 of cavity 230 may have a shelf portion width (W_{SP}) 252 and a shelf portion depth (D_{SP}) 258. In an example, shelf portion 232 of cavity 230 may be a cylindrical portion of the cavity corresponding to a cylindrical shelf 150 (e.g., where the shelf portion width (W_{SP}) is equivalent to the shelf portion diameter). Trunk portion 234 may be positioned between shelf portion 232 and cap portion 236 of barb cavity 230. Additionally, trunk portion 234 of cavity 230 may gradually decrease in diameter between shelf portion 232 and cap portion 236 at an angle (β). Additionally, the end of the trunk portion 234 adjacent cap portion 236 may have a trunk portion width (W_{TP}) 262 and a trunk portion depth (D_{TP}) 268. In an example, trunk portion 234 may be a cylindrical, conical, or frustoconical cavity corresponding to a cylindrical, conical, or frustoconical trunk 160 (e.g., where the trunk portion width (W_{TP}) is equivalent to the trunk portion diameter). Cap portion 236 may be positioned adjacent trunk portion 234 opposite shelf portion 232. The cap portion 236 of barb cavity 2300 may similarly have a cylindrical, conical, or frustoconical shape with a base of the cap portion and a top of the cap portion. For example, the cap portion 236 of cavity 230 may gradually decrease in diameter between the base of cap portion 236, which interfaces with trunk portion 234 and the top of the cap portion 236. For example, the base of the cap portion 236 may have a width (W_{CPB}) 272 and the top of the cap portion 236 may have a width (D_{CPT}) 274. In an example, the dimeter may change in a linear profile at an angle (β). Additionally, cap portion 236 may have a cap portion depth (D_{CP}) 278.

As illustrated in FIG. 5E, the barb cavity depth (D_{CAV}) 238 is equivalent to the sum of the shelf portion depth 258, the trunk portion depth 268 and the cap portion depth 278 (e.g., D_{CAV}=D_{SP}+D_{TP}+D_{CP}). In an example embodiment, the barb cavity depth (D_{CAV}) may be approximately equal to the barb height (H_{B}) 138. Similarly, the depth of the shelf portion 258 may be approximate to shelf height (H_{S}) 158, the depth of the trunk portion 268 may be approximate to trunk height (H_{T}) 168, and the cap depth of the cap portion 278 may be approximate to cap height (H_{C}) 178. Additionally, the shelf portion width 252 and the shelf width (W_{S}) 152, the trunk portion width 262 and the trunk width (W_{T}) 162, and the corresponding features of the cap and cap portion of barb cavity 230 may have approximately the same size, shape, and dimensions. In an example, angle (β) 276 may be approximately 120 degrees.

In an example, eversion tip(s) 120 (e.g., eversion tip 120a and eversion tip 120b) may have different sizes and/or contours. For example, eversion tip 120a may have a different size, shape, and/or contour than eversion tip 120b, which advantageously increases the versatility of the everter device 100 by permitting its use with a greater size range of vessels and/or anastomosis couplers. However, distal ends 210 of each eversion tip 120 may have the same profile so that each end of the everter device 100 may be used to pilot arteries and veins typically involved in microvascular anastomosis (e.g., arteries or veins with a diameter ranging from 1 mm to 4 mm). For example, eversion tip 120 may include a narrow distal end 210 to pilot down the bore of vessels, which may have a tendency to collapse in on themselves. The distal end 210 may be sized such that regardless of the overall size of eversion tip 120, the distal end 210 is adapted to be inserted into the smallest vessels (e.g., 0.1 mm or 0.5 mm diameter openings). After piloting through the end of the vessel, the flared contoured shaped of eversion tip 120 may provide support for the vessel wall as the eversion tip 120 is advanced through the vessel towards a ring coupler.

FIG. 5F and FIG. 5G illustrate example dimensions of eversion tip(s) 120 and the contour of eversion surface 220 and distal end 210. Each of the dimensions shown on FIG. 5F and FIG. 5G are in millimeters (mm) and illustrate example embodiments of eversion tip(s) 120. It should be appreciated that the eversion tip(s) 120 may have different sizes, shapes, contours, and geometry.

### Sizing Device

FIG. 6A illustrates an example embodiment of a sizing device 300. Sizing device 300 includes one or more sizing guides (e.g., sizing guide 320a for large vessels and sizing guide 320b for small vessels, hereinafter referred to generally as sizing guide 320) positioned on a handle 310. In an example, sizing guide 320a is positioned on a first end of handle 310 and sizing guide 320a is positioned on a second end of handle 310. Each

Handle 310 may be formed of a rigid or semi-rigid material, such as thermoplastic, and may be shaped similar to that of handle 110 to provide a similar feel and allow the user to similarly manipulate and move both the everter device 100 and sizing device 300. In an example, handle 310 may be made from MAKROLON 2458 polycarbonate.

As illustrated in FIGS. 6A, 6B, 6C and 6D, handle 310 may include gripping members 314a, 314b and 314c (similar to gripping member 114 of FIG. 1A). For example, the bottom of handle 310 may include gripping member 314a or a plurality of ribs or ridges to provide the user, such as a medical practitioner, additional gripping and handling features on handle 310. Additionally, gripping members 314b and 314c may comprise a plurality of projections or bumps (e.g., an omnidirectional dot pattern of bumps or protrusions at a height of approximately 0.012 inches (0.30 mm) to provide better gripping and handling characteristics of sizing device 300. It should be appreciated that other gripping means may be included on handle 310 (e.g., knurling, grips, ridges or the like) to provide additional gripping and handling characteristics to sizing device 300. The gripping members 314 may aid the user, such as a medical practitioner, when the user is handling and/or manipulating the sizing device 300.

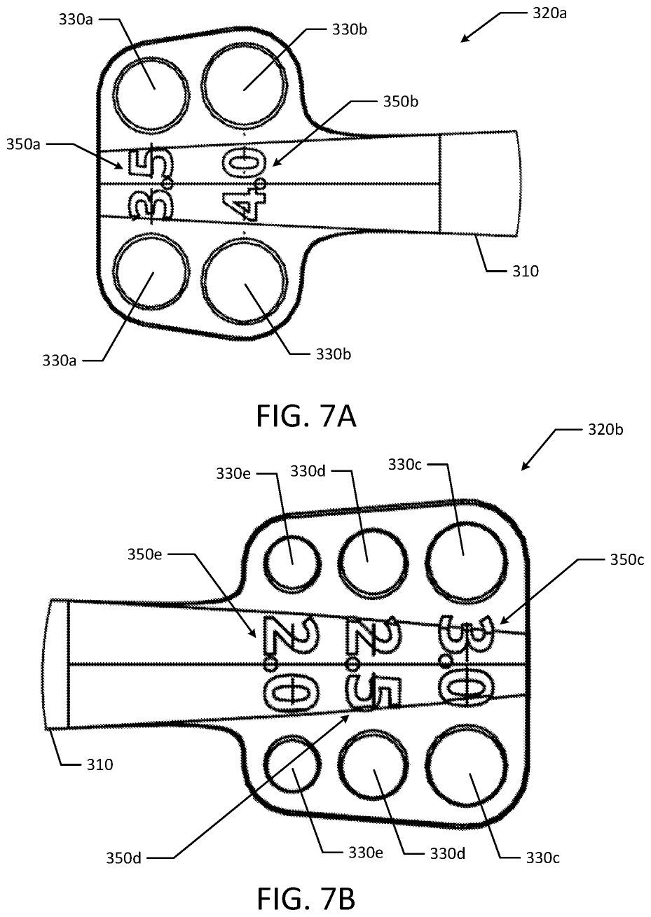

Each sizing guide 320 may include one or more sizing aperture 330 (as Illustrated in FIGS. 6D, 7A and 7B). For example, sizing guide 320a may include sizing apertures 330a and 330b. Similarly, sizing guide 320b may include sizing apertures 330c, 330d and 300e. As illustrated in FIGS. 6A, 7A and 7B, sizing apertures may be provided on sizing guide(s) 320 in pairs. For example, sizing guide 320a may include a pair of sizing guides 330a of the same diameter and a pair of sizing apertures 330b of the same diameter. Each sizing guide 320 may be associated with a sizing indicator 350. For example, pair of sizing guides 330a may be associated with sizing indicator 350a (e.g., “3.5” to indicate that the sizing guide 330a has a diameter of 3.5 mm), sizing guides 330b may be associated with sizing indicator 350b (e.g., “4.0” to indicate that the sizing guide 330b has a diameter) and so on. Similar, sizing guide 320b may include pairs of sizing guides 330c, 330d and 330e associated with sizing indicators 350c, 350d and 350e respectively. The sizing indicators 350 may be embossed or debossed. In another example, the sizing indicators 350 may be etched or painted on the handle 310. Sizing indicators 350 may also include other visual designations that indicate the diameter or size of sizing guides 330. Sizing indicators 250 may be colors (e.g., red, blue, green, etc.) associated with each sizing guide 330. For example, sizing guide 330a may have an outline of a first color (e.g., red) to indicate that sizing guide 330a has a diameter of 3.5 mm while sizing guide 330b is outlined in a second color (e.g., blue) to indicate that sizing guide 330b has a diameter of 4.0 mm. In an example, sizing guides 330 associated with the most common vein or artery sizes may be placed near the distal ends of sizing device 300 to provide easy access and improve ease of use for medical practitioners when sizing veins and/or arteries. For example, in some cases, vessel diameters of 3.0 mm and 3.5 mm may be the most common vessel diameter sizes encountered for a specific type of surgery and thus are placed at the distal most portion of sizing guides 320.

Sizing guides 320 may be formed as a single piece with handle 310. In an alternative example, sizing guides may be press-fit onto handle 310. For example, sizing guides 320 may be interchangeable for differing use cases depending on space restrictions and/or vessel diameters. Instead of two rows (e.g., pairs) of sizing apertures 330, a sizing guide 320 with a single row of sizing apertures 330 may be press-fit onto sizing device 300 to reduce the profile of sizing device 300 when sizing a vessel in a tight or hard to reach area during surgery. Additionally, a sizing guide 320 with larger or smaller variations in sizes may be used (e.g., with vessel diameter increments of 0.10, 0.25, 0.5, 1.0, etc.). In the above alternative example, handle 310 may include barbs, similar to barb(s) 130 of FIG. 2A, which are sized and shaped such that it retains a removable and/or interchangeable sizing guide 320 on handle 310. For example, sizing guide(s) 320 may be press-fit onto barb(s) of handle 310 such that they are removably retained on handle 310. In an example, the sizing guide(s) 320 may be rotatably coupled to handle 110 via barb(s) 130.

Sizing guides 320 may be angled upwards from the bottom of handle 310 at an angle (λ) 332. The angled orientation of sizing guides 320 may be adapted to assist with positioning vessels through the apertures 330 of the sizing guide at both ends. For example, by providing sizing guides 320 an angle 332, a medical practitioner may route vessels through apertures 330 on each side of the sizing guide 320 so that each vessel can be positioned on a coupler ring. In an example, angle (λ) 332 is approximately 20 degrees. In another example, the angle (λ) 332 may be approximately 25 degrees to 45 degrees to provide a smoother transition for each vessel routed through an aperture 330 on the sizing guide 320. Additionally, the sizing device 300 may be shaped and arranged such that the sizing guides 320 mimic the shape and structure of a coupler ring and to provide lateral access to position vessels on respective coupler rings.

### Eversion Device and Sizing Device Kit

The eversion device 100 and sizing device 300 may be provided in a kit. The kit may be co-pouched, such that each of the eversion device 100 and sizing device 200 are placed in a first pouch, which is then placed in another outer pouch. Each pouch may be made from Poly-Tyvek. In an example, the Poly-Tyvek may be Tyvek® 1073B. Additionally, the kit may be double sterilized using Ethylene Oxide (ETO) sterilization, which advantageously prevents cross-linking within the eversion tip 120.

### Vessel Eversion and Attachment to Coupler Ring

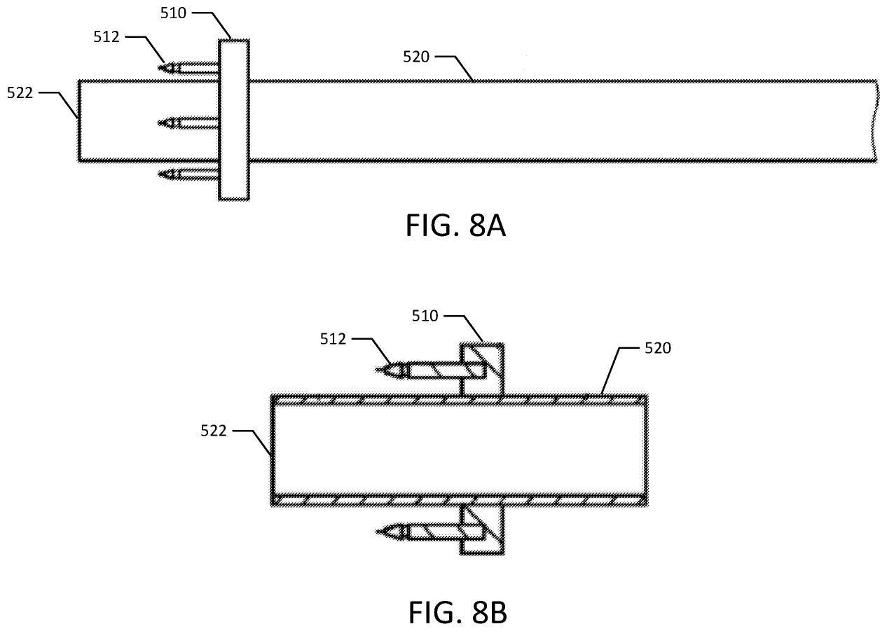

As illustrated in FIGS. 8A and 8B a coupler ring 510 is provided near a free end 522 of a vessel segment 520 that is to be surgically coapted to another vessel segment (not shown) using microanastomosis. The coupler ring 28 is arranged with its pins or posts 512 directed toward the free end 522. In an example, the vessel segment 520 may be part of an artery or vein that has been clamped by a vessel clamp 560 (as illustrated in FIG. 9A) upstream of or behind the coupler ring 510. Throughout the procedure, free end 522 of vessel segment 520 may be irrigated.

Prior to selecting and positioning coupler ring 510 on vessel segment 520, sizing device 300 may be used to determine the size of each vessel segment 520 that is to be surgically coapted together. For example, the vessel segment 520 may be positioned through various sizing apertures 330 of sizing guide 320 until the vessel diameter is determined. If the vessel side walls are pinched, crimped, creased, or pleated when placed in a sizing aperture 330, then the vessel is larger than the selected sizing aperture 330 and can be positioned through the next largest aperture until no more pinching, crimping, creasing and/or pleating is visible. Once the diameter of the vessel segment 520 is determined, an appropriate coupler ring 510 may be selected and positioned about vessel segment 520 as illustrated in FIGS. 8A and 8B. For example, a coupler ring designed for a 4.0 mm vessel diameter may be selected after the medical practitioner confirms the vessel diameter is approximately 4.0 mm from the sizing device 300.

After the coupler ring 510 has been positioned on the vessel segment 520 near the free end 522 of vessel segment 520, as illustrated in FIGS. 9A and 9B, the everter device 100 is advanced toward the vessel segment 520 until at least a portion of eversion tip 120 is received in the vessel segment 520. In an example, everter device 100 is advanced towards and into vessel segment 520 until the distal end 210 of the eversion tip 120 extends just beyond the coupler ring 510.

In an effort to simultaneously impale all of the pins or posts 512 of the coupler ring 510 through the free end 522 of the vessel segment 522, the eversion tip 120 is advanced into vessel segment 522 until the eversion surface 220 of eversion tip 120 contacts the free end 522 of vessel segment 520. The eversion device 100 is further advanced toward the coupler ring 510 to cause the vessel segment 520 to evert thereby causing pins or posts 512 of the coupler ring 510 to pierce through the vessel wall tissue of the free end 522 of the vessel segment 520. In an example, the user (e.g., medical practitioner) applies sufficient force to the everter device 100 in the direction of the coupler ring 510 to cause the pins or posts 512 of the coupler ring 510 to pierce through the vessel wall tissue. As discussed above, upon piercing through the vessel wall tissue, the pins or posts 512 may also pierce through eversion tip 120, as illustrated in FIG. 9B. With the free end 522 of the vessel segment 520 secured to the coupler ring 510, the everter device 110 may be withdrawn from vessel segment 520.

When advancing the eversion tip 120 through the vessel and applying force to the everter tool 100, the eversion tip 120 advantageously rotates freely about retention barb 130 to prevent the everter tool 100 from damaging pins or posts 510 of coupler ring 510. For example, if the eversion tip 120 did not rotate, rotation of everter tool 100 by the medical practitioner may cause a pin or post 510 to bend or break and therefore would be unable to be joined with a mating coupler ring.

Specifically, when the everter device 100 is sufficiently advanced toward the free end 522 of vessel segment 520 so as to contact the vessel tissue, the eversion tip 120 is further advanced toward the coupler ring 510, with the contoured eversion surface 220 everting the free end 522 of vessel segment 520, as illustrated in FIG. 9B. The contour of eversion surface 220 maintains the shape of the arterial vessel and prevent the free end 522 from bucking inward without damaging the vessel wall's intima. By maintaining the shape of the vessel, the contour of eversion surface 220 permits a substantially continuous application of force annually along the coupler ring 510 to offset the tendency of the relatively thick arterial tissue to recover its natural shape and lose engagement with the pins or posts 512 of the coupler ring 510 as the vessel tissue is everted and secured to the coupler ring 510. As the everter device 100 is pushed further forward, the vessel wall is flared out circumferentially over the pins or posts 512 of the coupler ring 510. The eversion tips 120 may also deform to assist with vessel eversion as the deformation causes the eversion tip 120 to compress slightly and “balloon” or “mushroom” out thereby further pressing against the internal vessel wall and causing the vessel to further evert.

Typically, arteries are more muscular or more difficult to deform and evert as compared to veins and the above described everter device 100 and anastomotic coupling method are particularly suited for arterial connections. For example, as discussed above, the everter device advantageously maintains the shape of the arterial wall and “balloons” or “mushrooms” to further assist with arterial eversion.

As discussed above, each of the pins (e.g., all six pins) may preferably and simultaneously (or nearly simultaneously) pierce through the vessel wall and into the outer wall of the eversion tip 120. In an example, the eversion tip 120 may deform as it comes into contact with the coupler ring 510, thereby further sliding the vessel down the pins or posts 512.

As illustrated in FIG. 9A, microvascular clamp 560 may be clamped to the vessel segment 520 upstream of the coupler ring 510. The vessel clamp or microvascular clamp 560 may prevent the vessel segment 520 from sliding back through the coupler ring 510 thereby allowing everter device 110 to evert free end 522 of vessel segment 520.

The above procedure is repeated on another vessel segment (not shown) to be coapted to the first vessel segment 520, so as to secure a free end of that other vessel segment to a mating coupler ring (also not shown). After both vessel segments are attached to coupler rings (e.g., coupler ring 510), the coupler rings may be joined to complete the end-to-end microanastomosis.

As explained above, the everter device 100 may be disposable. Additionally, everter device 100 may be designed for one procedural use (e.g., used to evert one or more arteries or veins during a surgical procedure). For example, everter device 100 may be used several times during a procedure before being disposed of.

### Alternative Embodiments

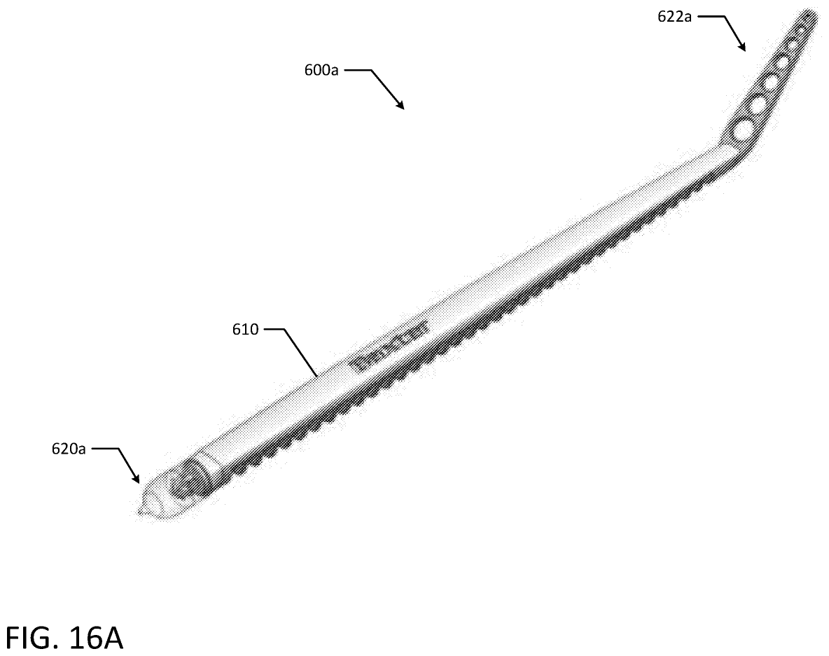

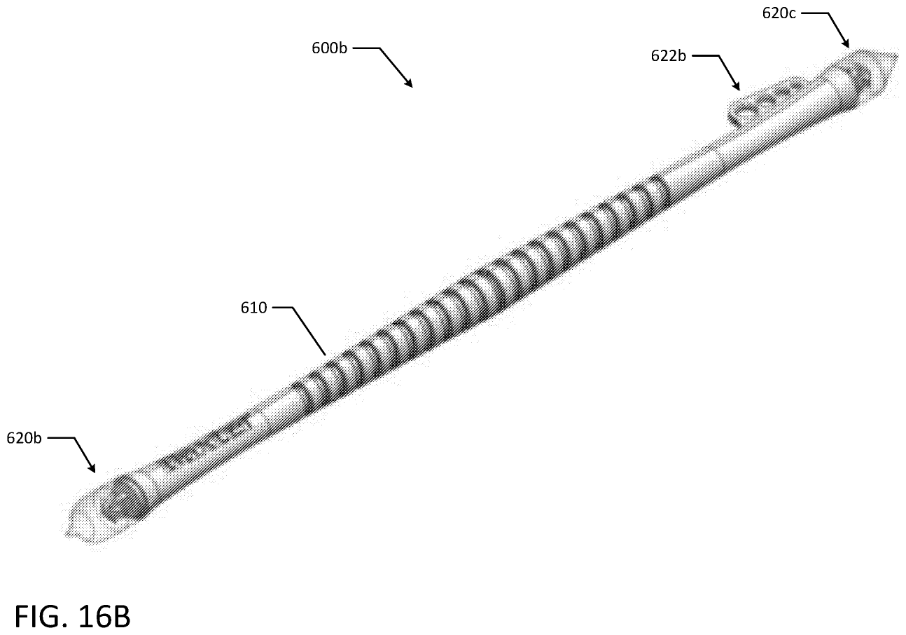

FIG. 16A illustrates an alternative embodiment of a combination everter and sizing device 600a. In the alternative embodiment, the combination everter and sizing device 600a includes an eversion tip 620a at one end of handle 610a and a sizing guide 622a at the other end of handle 610a. Sizing guide 622a may be similar to sizing guide 320 discussed above. Similarly, eversion tip 620a may be similar to eversion tip 120 discussed above. Additionally, handle 610a may include any of the features discussed above with respect to handle 110 and/or handle 310. In the illustrated alternative embodiment, sizing guide 622a may include a single row of sizing apertures. It should be appreciated that other layouts and orientations of sizing apertures may be used.

FIG. 16B illustrates another alternative embodiment of a combination everter and sizing device 600b. In the alternative embodiment, the combination everter and sizing device 600b includes two eversion tips 620b and 620c, similar to that of sizing device 100 (illustrated in FIGS. 1A-C) and a sizing guide 622b at the other end of handle 610b. Sizing guide 622b may be similar to sizing guide 320 discussed above. Similarly, eversion tips 620b and 620c may be similar to eversion tip(s) 120 discussed above. Additionally, handle 610b may include any of the features discussed above with respect to handle 110 and/or handle 310. In the illustrated alternative embodiment, sizing guide 622b may include a single row of sizing apertures positioned on a lateral size of handle 610. It should be appreciated that other layouts and orientations of sizing apertures may be used.

### Design Optimization

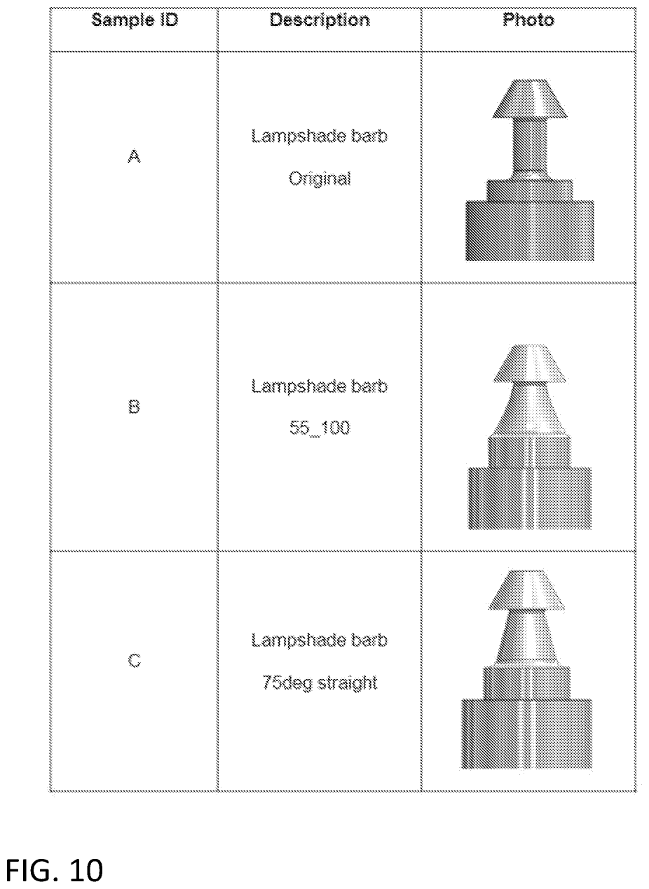

Retention Barb Yield Strength Test: During use, the everter device 100 may experience rocking around its center axis (e.g., axis 105 along the length of handle 110 and through center of each retention barb 130). The “worst case scenario” was tested by applying a compressive load perpendicular (e.g., 90 degrees) to the center axis 105 of retention barb 130. In the study, retention barb 130 was analyzed without eversion tip 120 in place in order to isolate and compare retention barb mechanical strengths between various retention barb designs. Three retention barb designs, as illustrated in FIG. 10, were analyzed (e.g., “original lampshade”-design_A, “lampshade 55_100”-design_B, and “lampshade 75deg straight”-design_C), which were chosen based on results from finite element analysis (“FEA”) tests of various retention barb designs. The FEA tests determined that “lampshade 55_100” and “lampshade 75deg straight” performed the best in terms of yield strength, and thus these designs were selected to be included in the physical force testing alongside the “original lampshade” design.

Each of the designs in FIG. 10 were machined from Protolabs polycarbonate resin and each retention barb 130 was subjected to a compressive load until failure is reached (e.g., retention barb tip breaks off). The loading rate was set at 20 mm/min at a sampling frequency of 10 Hz. The average ultimate yield strength for each design was determined with design_A, deign_B, and design_C achieving average ultimate yield strengths of 4.08 (lbf), 15.54 (lbf), and 14.74 (lbf) respectively. Each of the retention bar designs were able to withstand the “worst case scenario” of a compressive force of 2 lbf loaded normally to the retention barb 130 without the eversion tip 120 in place, however as noted above, design_B and design_C both performed approximately three times better than design_A in terms of ultimate yield strength.

Retention Barb Rocking Test: When the eversion tip 120 is pulled upwards, a tensile force is created on the retention barb 130. Additionally, the device may be misused by the user while rocking the eversion tip 120 around the center axis in order to secure the vessel on the coupler ring 510. Therefore, testing was performed for a “worst case scenario” of a 45 degree or 90 degree load being applied to the retention barb 130. Furthermore, two materials were tested (ABS and PC).

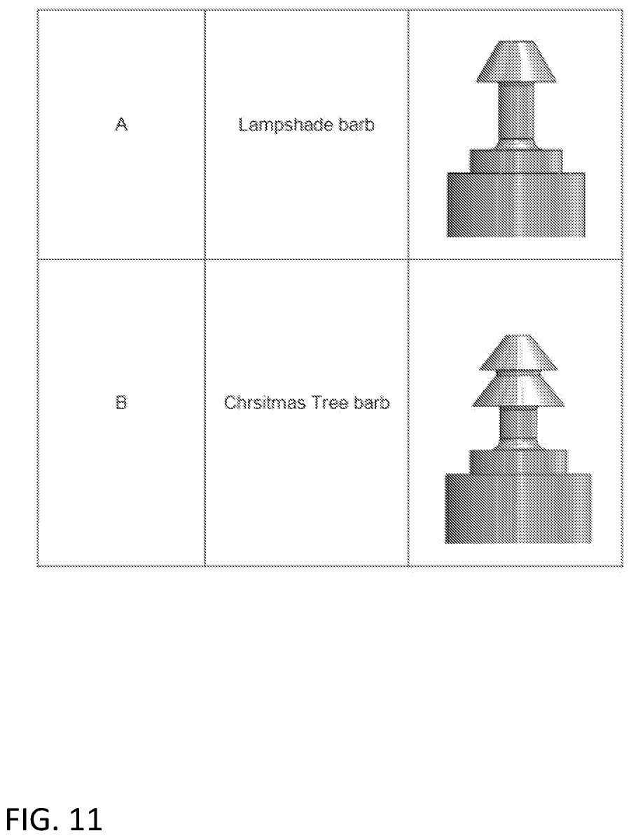

Each design illustrated in FIG. 11 (e.g., design A for “lampshade barb” and design B for “Christmas tree barb”) was tested under a 11b, 21b, 51b, 101b and 201b axial tensile force. Additionally each design was tested under a 11b, 21b, 51b and 101b 45 degree compressive force. Furthermore, each design was tested under a 11b, 21b, 51b and 101b 90 degree compressive force. Tests were simulated using FEA and SolidWorks default material properties were used. The results showed that retention barbs made of PC material faired better than ABS as PC material has a higher yield strength. However, both materials and designs (“lampshade barb” and “Christmas tree barb”) failed at or below 21b.

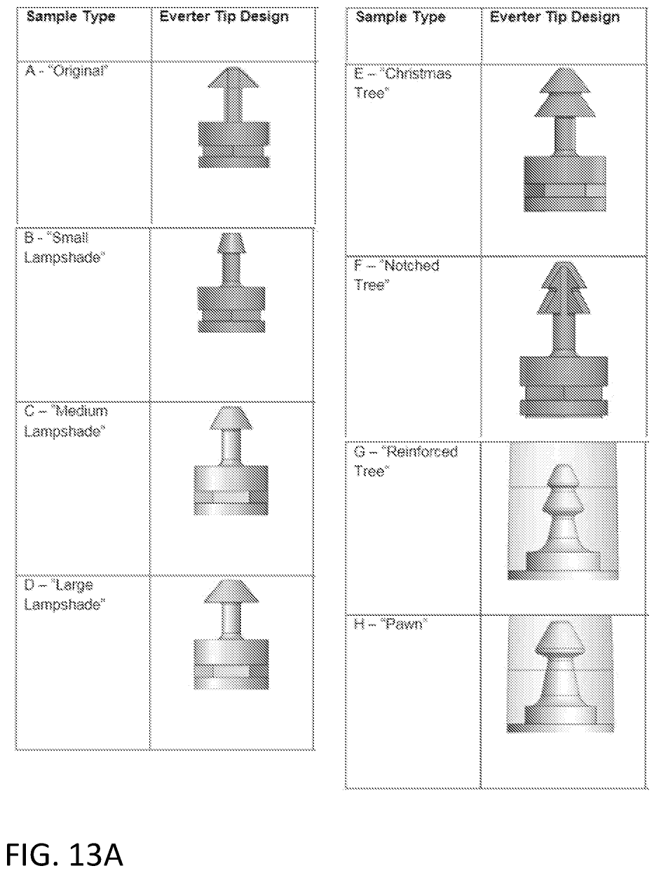

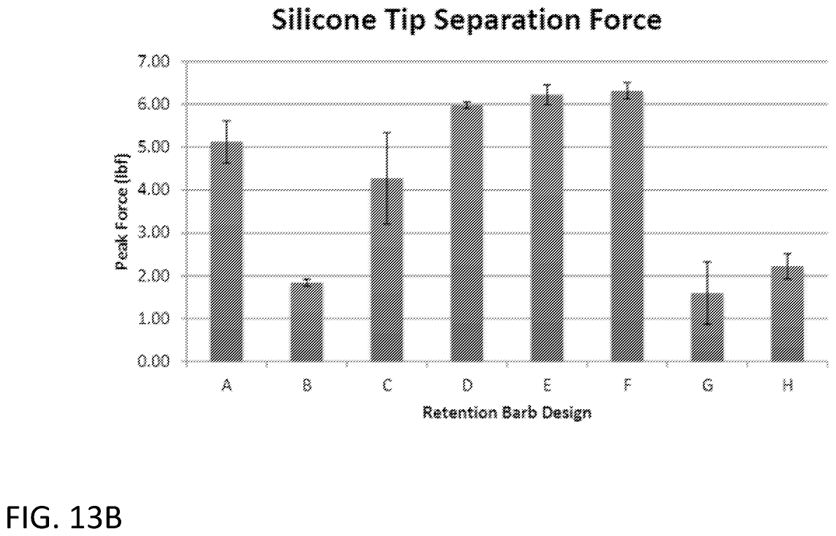

Retention Barb Geometry/Strength Test 1: The purpose of this study is to evaluate various retention barb features for which one or ones have the greatest tensile strength. For this testing, eight retention barb geometries were evaluated as illustrated in FIG. 13A. Each retention barb protruded from an identical shortened hexagonal shaft. The silicone eversion tips 120 were molded into a long cylindrical shape to facilitate gripping during tensile testing.

Each handle 110 was placed in vice grips and the silicone eversion tip 120 was grasped with Mark-10 wedge grips at a location at or above the location of the distal end of the internal retention barb 130. Then, each eversion tip 120 was then pulled upwards away from the handle 110 at a rate of 150 mm/min.

Both of the “F” design (e.g., notched tree) samples that were tested tore instead of cleanly separating. Additionally, the retention barb 130 broke during separation on one design “A” and one design “D” sample. All other samples had a clean retention barb/silicone separation. The average peak force for silicon tip separation for each retention barb design is shown in FIG. 13B, with standard deviation bars included, which represents the retention strength for reach retention barb 130 design of FIG. 13A.

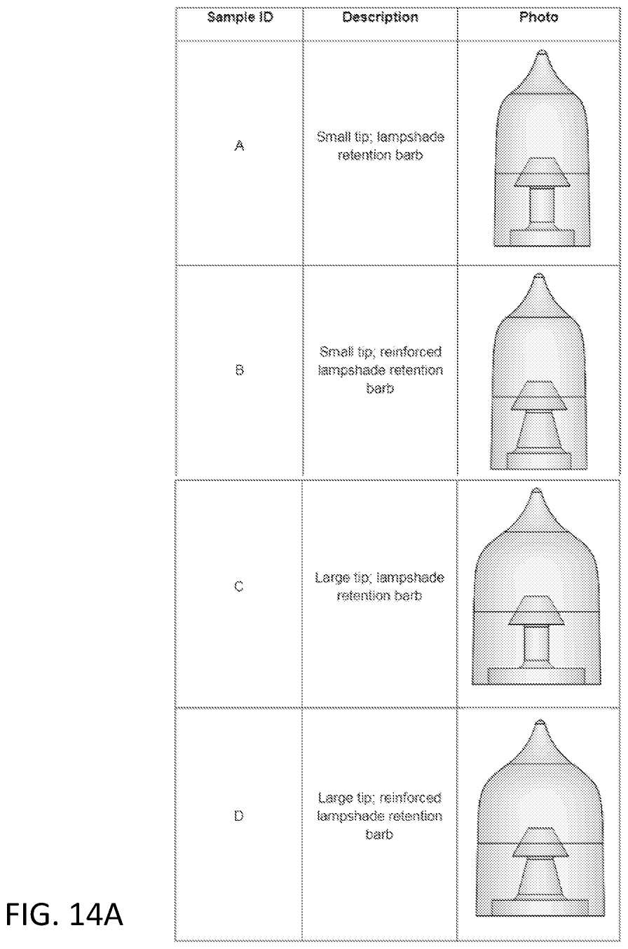

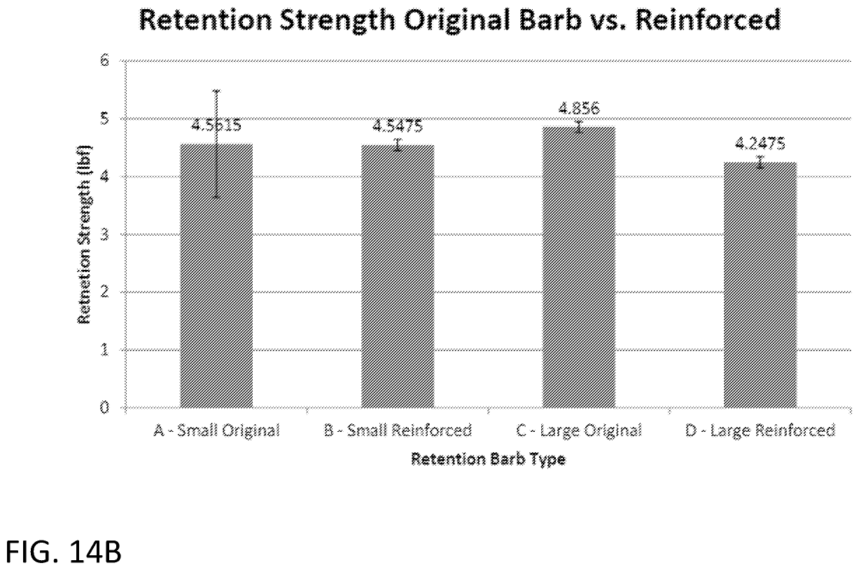

Retention Barb Geometry/Strength Test 2: The purpose of this study was to compare the retention strength of the reinforced retention barb 130 to the original retention barb 130. Updated retention barb geometries were evaluated as illustrated in FIG. 14A. Since the previous round of retention force testing (e.g., Retention Barb Geometry/Strength Test 1), the design of “lampshade” retention barb has been modified to increase its resistance to cantilever loading in a direction perpendicular to the access of the Everter. This modification was performed on both the small and large lampshade and is referred to as the “reinforced lampshade.”

The same testing parameters as Retention Barb Geometry/Strength Test 1 were used and resulted in the average peak force for silicon tip separation or average retention strength shown in FIG. 14B. The testing showed that there was no significant difference in retention strength between the reinforced retention barb design and the original retention barb design. The reinforced barb is significantly stronger when subjected to cantilever loading than the original barb. Therefore, retention barb geometries similar to the reinforced designs (“B” and/or “D”) with sloped trunk sections provide improved retention properties.

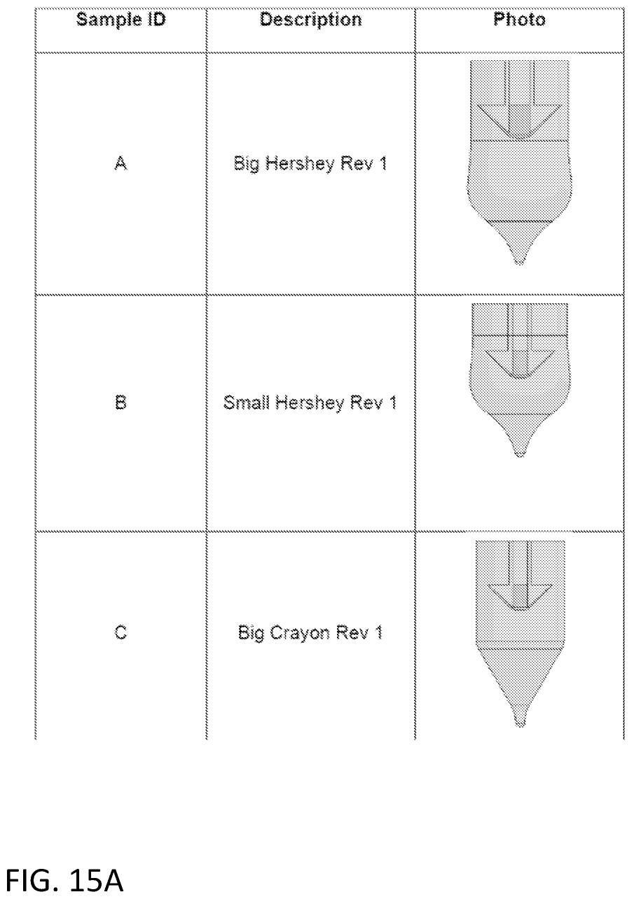

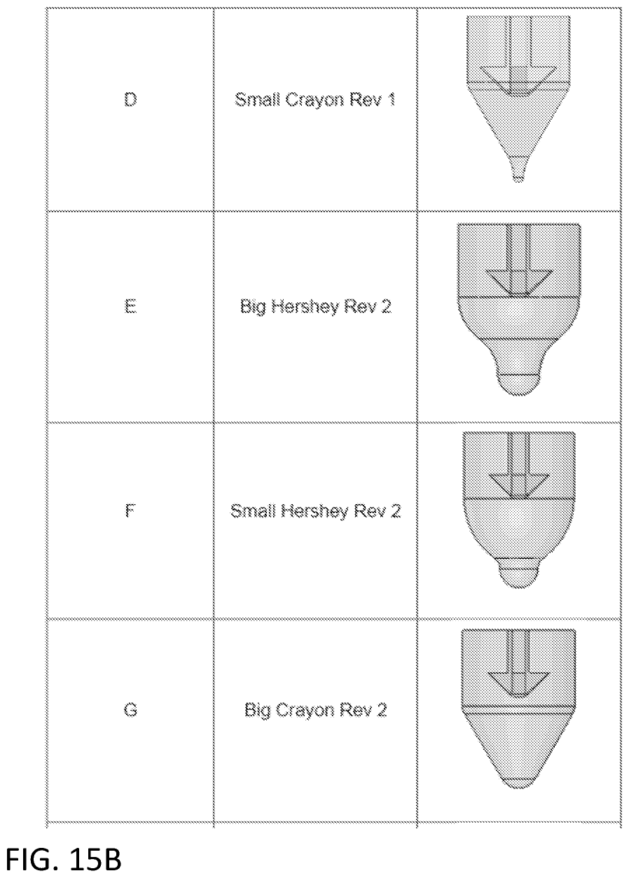

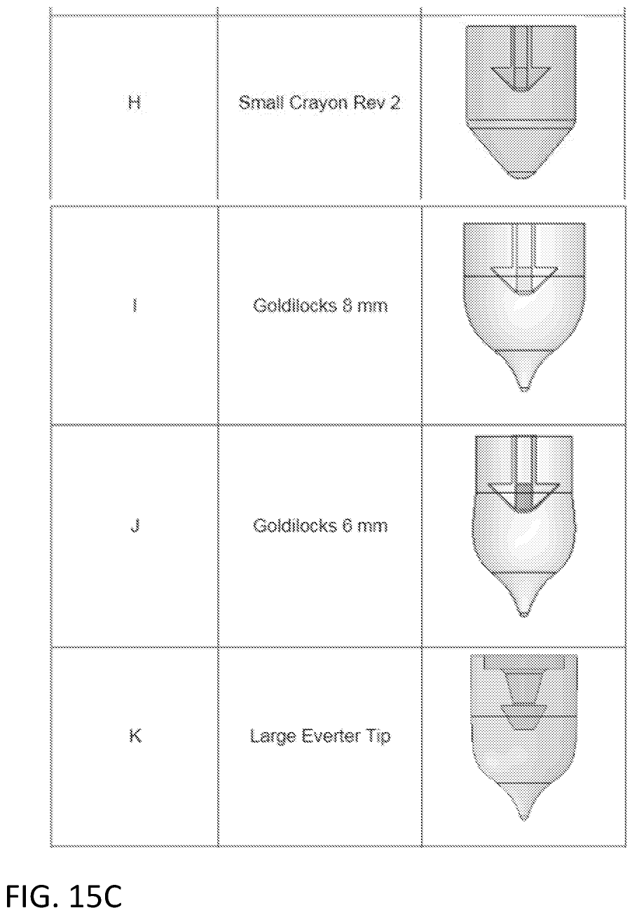

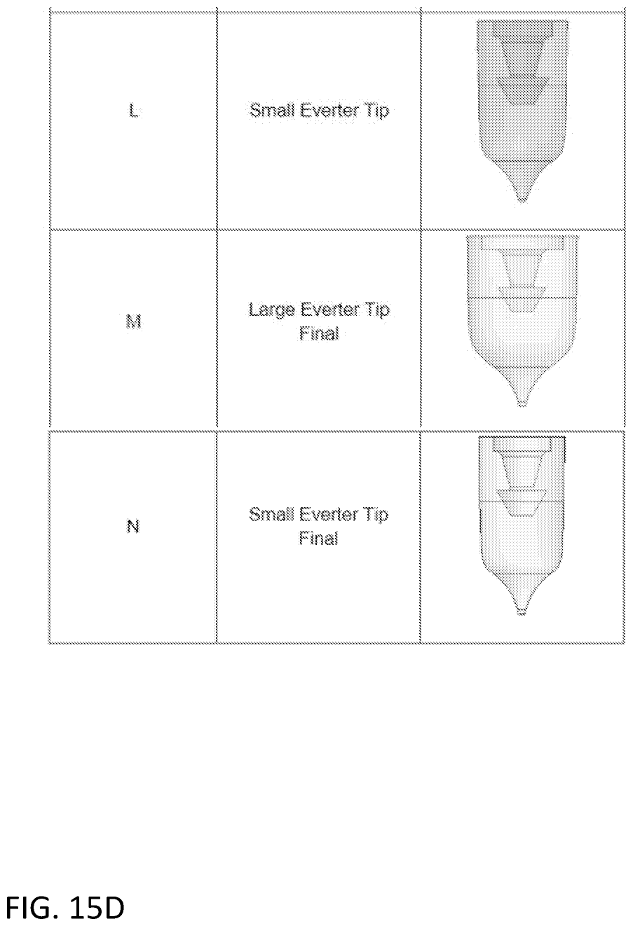

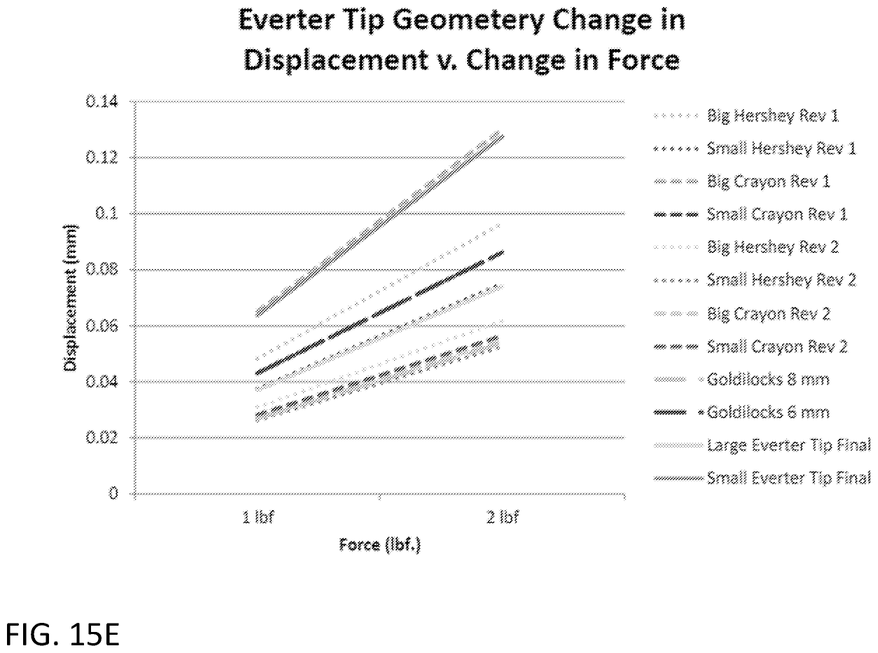

Eversion Tip Geometry Test: The purpose of this study was to evaluate the functionality of fourteen different silicone eversion tip geometries, as illustrated in FIGS. 15A to 15D, when an axial compressive force is applied at the distal end 210 of the eversion tip 120. As the everter device 100 is pushed into the coupler ring 510 there is an axial compressive force applied to the silicone eversion tip 120. Furthermore, as the silicone eversion tip 120 is compressed, it is preferred that the silicone eversion tip 120 “balloon” or “mushroom” so that the vessel is seated properly onto the pins 512 of the coupler 510. In order to determine which silicone eversion tip 120 compresses in the desired manner, yet is still able to withstand the loading scenarios, an FEA test was conducted to determine optimum eversion tip geometries. The different silicone eversion tip geometries were tested at an axial compressive force of 0.5lbf., 1.0lbf., 1.5lbf, and 2.0lbf., which are compressive forces associated with anticipated loading scenarios.

The material of the silicone eversion tip 120 in Solidworks was modeled to mirror the material properties from the data sheet, MED-4840. The fourteen different silicone eversion tip geometries (A, B, C, D, E, F, G, H, I, J, K, L, M, and N) were tested using silicone material properties each under an axial compressive force of 0.5lbf., 1.0lbf., 1.5lbf., 2.0lbf.

A split line was created right below the maximum diameter of the silicone eversion tip to provide a reference plane when applying compressive loads during the FEA analysis. Due to the characteristics of eversion tip 120, all of the direct force from the coupler and artery/vessel will be translated onto the eversion tip 120 between the most distal end 210 and the split line.

For the Silicone eversion tip 120 of this study, the values used from the data sheet are tensile strength (1180 psi) and mass density (0.0405 lb/in³). The remaining material properties were obtained from the article “Overview of materials for Silicone Rubber.” The elastic modulus (3454 psi), Poisson's Ratio (0.47), shear modulus (3315.99 psi), compressive strength (10.7 psi) and yield strength (263 psi) were all added based off of the article, “Overview of materials for Silicone Rubber.” In the article a range of values were given and the chosen value was determined by using a proportion. Based on the tensile strength from the data sheet being 1180 psi and the maximum tensile strength being 94300 psi in the article the remaining values were found based on that proportion. Ex. 1180/94300=(elastic modulus)/276000, so the elastic modulus was set at 3454 psi.

Overall, based on the FEA test results and other contributing factors such as vessel eversion and manufacturability it was determined that the “Large Everter Tip Final (M)” and the “Small Everter Tip Final (N)” were the most effective and realistic eversion tips geometries in this study. Tip geometries “A” through “D” were the first revision, but these eversion tips 120 lacked the ability to evert larger vessels (3 mm+). The eversion tip 120 design was then updated to tip geometries “E” through “H”, which were able to evert some larger vessels because of the broader 8 mm shoulder, but because of the blunt distal tip geometries “E” through “H” were sometimes unable to pilot the vessels. The bulb-shaped cross section (e.g., designs “A”, “B”, “E” and “F”) captured more pins 512 than the crayon-shaped cross section (e.g., designs “C”, “D”, “G” and “H”) from previous evaluations. The designs were then consolidated to make eversion tip 120 geometries “I” and “J”. These designs have different shoulder widths, which are optimized to evert vessels small and large, an identical distal tip curvature so either side is able to pilot the smallest of vessels, and an optimized distance from the end of the barb to the end of the tip for best control and safest eversion of vessels. Though revisions “I” and “J” were able to evert and pilot the vessel, further revisions were made to “I” and “J” for manufacturability purposes, which resulted in eversion tip geometries “M” and “N”. Eversion tip geometries “M” and “N” are able to be manufactured along with being able to evert and pilot the vessel. Furthermore, both eversion tip geometries, “M” and “N”, showed an acceptable amount of distal tip displacement, which can be seen in FIG. 15E.

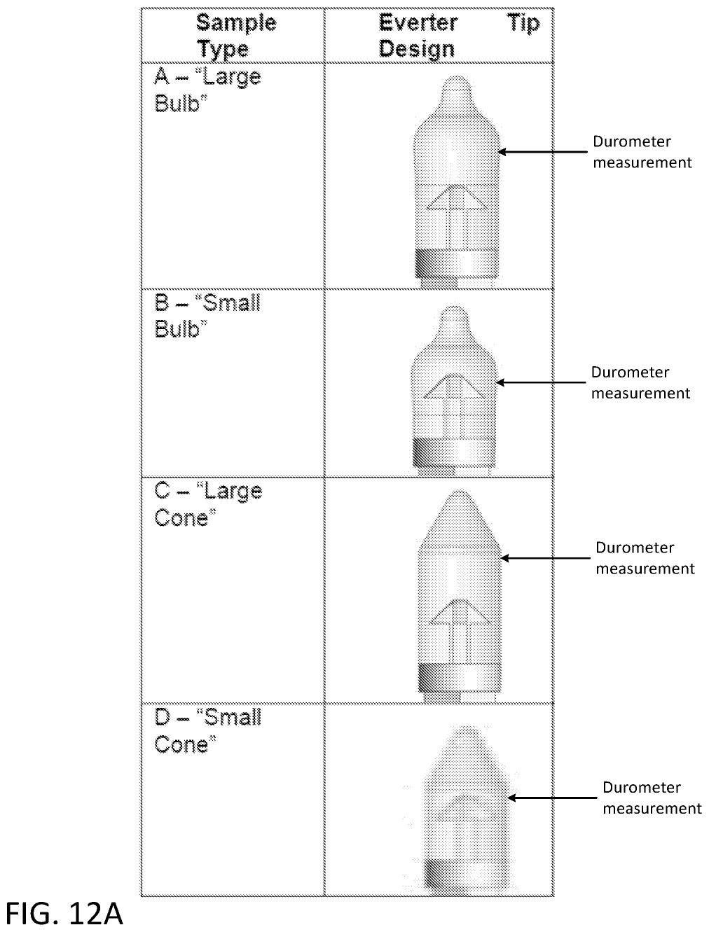

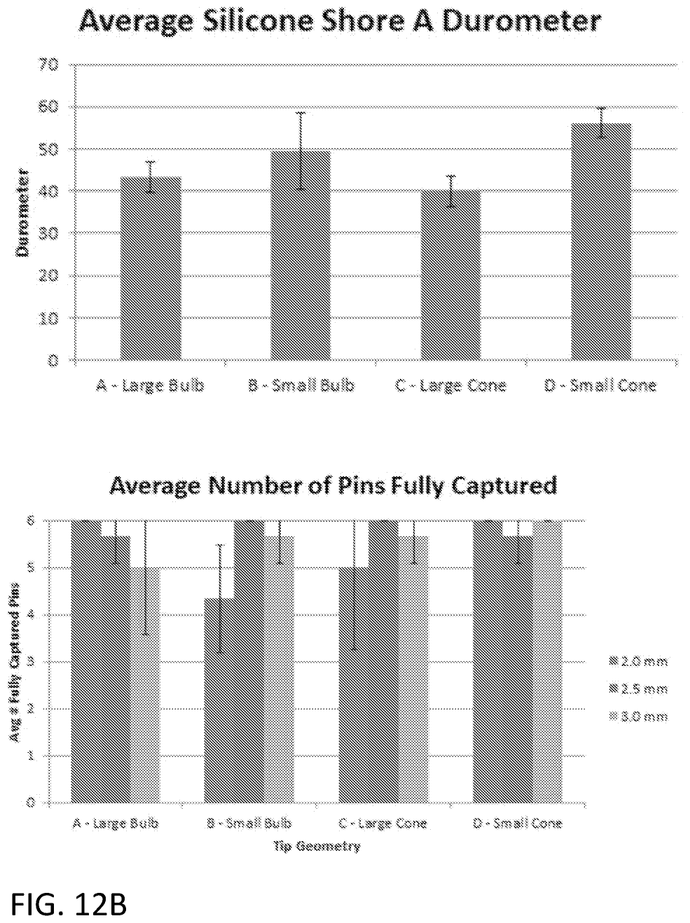

Eversion Tip Eversion Test: The eversion tip eversion test evaluated the performance of various eversion tip geometries, as illustrated in FIG. 12A, for everting vessels of various sizes. The performance of each eversion tip design was assessed according to the number of fully captured pins or posts 512, partially captured pins or posts 512, and non-captured pins or posts 512 on the coupler ring 510 upon initial application of the eversion tip 120.

Each of the design geometries was formed from Silicone from a 9:1 mixture of Shore A Polytek TinSil 80-40 Silicon Rubber Part A and Polytek TinSil 80-Series Silicon Rubber Part B. The testing was performed on porcine internal cranial mammary arteries cut into 2.5 cm segments, sorted by outer diameter into three groups: 2.0 mm, 2.5 mm and 3.0 mm. Additionally, the procine arteries were coated with saline to maintain moisture.

A portion of each vessel, approximately 1.75 times the length of the pins or posts 512, was drawn through the back side of each coupler ring 510. Then, eversion tip 120 was axially inserted into the opening of the vessel 520 until resistance was felt. Then, the eversion tip 120 was angled 45 degrees from the central axis of the artery and a complete 360 degree rotation about the artery and coupler ring 510 was completed. The eversion tip 120 was then straightened along the vessel's axis and them removed. The number of fully captured pins or posts, partially captured pins or posts, and non-captured pins or posts were then counted.

Additionally, the Shore A Durometer hardness of each tip design was measured by holding a durometer gauge normal to the surface of the silicone eversion tip, just before the tip began to taper as indicated by the “durometer measurement” arrow in FIG. 12A. The graphs in FIG. 12B illustrate the results of each of the designs for capturing pins or posts as well as hardness values.

The testing showed that all tip geometries performed well. Across all sample types, 27 out of the 36 samples (75%) fully captured all six coupler pins 512. There was only once incidence in which fewer than three pins were fully captured. In this incidence, the coupler popped out of the coupler applicator and two pins were still fully captured. There was no significant difference in functionality across the various designs. It was noted by the engineer performing the testing that he felt more “control” using the shorter tip designs (“B” and “D”).

The many features and advantages of the present disclosure are apparent from the written description, and thus, the appended claims are intended to cover all such features and advantages of the disclosure. Further, since numerous modifications and changes will readily occur to those skilled in the art, the present disclosure is not limited to the exact construction and operation as illustrated and described. Therefore, the described embodiments should be taken as illustrative and not restrictive, and the disclosure should not be limited to the details given herein but should be defined by the following claims and their full scope of equivalents, whether foreseeable or unforeseeable now or in the future.

1. An everter device comprising:
a handle having a first end and a second end; and
at least one eversion tip rotatably coupled to the handle, wherein each of the at least one eversion tip is coupled to a respective end of the handle, the at least one eversion tip having a respective distal end, a respective proximal end, and a respective eversion surface, wherein the eversion surface is a curved surface between the distal end and the proximal end of the eversion tip.

2. The everter device of claim 1, wherein the at least one eversion tip is removably coupled to the handle.

3. The everter device of claim 1, wherein the curved surface slopes outward toward a shoulder of the eversion tip and then slopes back inward after the shoulder as the curved surface approaches the proximal end.

4. The everter device of claim 1, wherein the eversion surface includes:
a piloting region starting at the distal end of the eversion tip;
a concave region;
a transition region; and
a nearly linear region ending at the proximal end of the eversion tip, wherein
the concave region connects the piloting region to the transition region,
the transition region connects the concave region to the nearly linear region,
the eversion surface transitions from a concave surface to a convex surface in the transition region,
the eversion tip has a diameter,
the diameter of the eversion tip in the concave region increases exponentially as the eversion surface approaches the transition region,
the diameter of the eversion tip in the transition region continuously increases through the transition region towards the nearly linear region,
the rate of change of the diameter of the eversion tip in the transition region continuously decreases through the transition region towards the nearly linear region, and
the diameter of the eversion tip in the nearly linear region continues to increase until reaching the proximal end of the eversion tip.

5. The everter device of claim 3 or 4, wherein the eversion surface is configured to expand radially outward at the shoulder in response to a compressive force applied on the curved surface, wherein the compressive force is applied toward the proximal end.

6. The everter device of claim 1, wherein the eversion tip is made from an elastic material, which is piercable, deformable, or that is adapted to axially recede to accommodate posts or pins of a coupler ring of an anastomosis clamp system.

7. The everter device of claim 6, wherein the elastic material is configured to deform or axially recede when the everter device is advanced into contact with posts or pins of a coupler ring.

8. The everter device of claim 6, wherein the elastic material is a thermoplastic elastomer.

9. The everter device of claim 8, wherein the thermoplastic elastomer is a silicone elastomer.

10. The everter device of claim 9, wherein the silicone elastomer is NUSIL 4840 silicone.

11. The everter device of claim 1, wherein the eversion tip has a Shore A hardness between 10 and 50.

12. The everter device of claim 1, wherein the eversion tip has a Shore A hardness between 30 and 45.

13. The everter device of claim 1, wherein the handle includes a plurality of gripping members.

14. The everter device of claim 1, wherein the handle includes at least one retention barb configured and arranged to retain the at least one eversion tip on the handle.

15. The everter device of claim 14, wherein the retention barb includes a shelf, a trunk and a cap, wherein the trunk is adjacent to the shelf and extends from the shelf to the cap, and wherein the cap forms a respective end of the handle.

16. A sizing device comprising:
a handle having a first end and a second end; and
at least one sizing guide, wherein each of the at least one sizing guide is coupled to a respective end of the handle, the at least one sizing guide having a plurality of sizing apertures and associated sizing indicators that indicate a diameter of the respective at least one sizing guide.

17. The sizing device of claim 16, wherein the at least one sizing guide is removably coupled to the handle.

18. The sizing device of claim 16, wherein the handle includes a plurality of gripping members.

19. The sizing device of claim 16, wherein the handle includes at least one retention member configured and arranged to retain the at least one sizing guide on the handle.

20. The sizing device of claim 16, wherein the retention member is a retention barb.

21. The sizing device of claim 16, wherein the plurality of sizing apertures have diameters between 0.1 mm and 10.0 mm.

22. An eversion kit comprising:
an everter device including:
a first handle having a first end and a second end, and
at least one eversion tip rotatably coupled to the first handle, wherein each of the at least one eversion tip is coupled to a respective end of the handle, the at least one eversion tip having a respective distal end, a respective proximal end and a respective eversion surface, wherein the respective eversion surface is a curved surface between the respective distal end and the respective proximal end of a respective eversion tip of the at least one eversion tip; and a sizing device including:
a second handle having a first end portion and a second end portion, and
at least one sizing guide, wherein the at least one sizing guide is coupled to a respective end portion of the handle, the at least one sizing guide having a plurality of sizing apertures and associated sizing indicators that indicate a diameter of the respective at least one sizing guide.

23. The eversion kit of claim 22, wherein the at least one eversion tip of the everter device is removably coupled to the handle.

24. The eversion kit of claim 22, wherein the curved surface slopes outward toward a shoulder of the eversion tip and then slopes back inward after the shoulder as the curved surfaces approaches the proximal end of the eversion tip.

25. The eversion kit of claim 22, wherein the eversion tip is made from an elastic material, which is piercable, deformable, or that is adapted to axially recede to accommodate posts or pins of a coupler ring of an anastomosis clamp system.

26. The eversion kit of claim 22, wherein the eversion tip has a Shore A hardness between 10 and 50.

27. The eversion kit of claim 22, wherein the eversion tip has a Shore A hardness between 30 and 45.

28. The eversion kit of claim 22, wherein the first handle and the second handle both include a plurality of gripping members.

29. The eversion kit of claim 22, wherein the first handle includes at least one retention barb configured and arranged to retain the at least one eversion tip on the first handle.
